(19) United States
(12) Patent Application Publication
(10) Pub. No.: US 2025/0135027 A1
(43) Pub. Date: May 1, 2025

(54) BEADS FOR TARGETED SIGNAL DELIVERY

(71) Applicant: RUBHU BIOLOGICS, INC., Athens, GA (US)

(72) Inventors: Surendra Jagannath CHAVAN, Lilburn, GA (US); William Carson Hurst SEAWELL, Hoschton, GA (US); Madusha Lakshani WATUTHANTHRIGE PERERA, Athens, GA (US)

(21) Appl. No.: 18/694,212

(22) PCT Filed: Sep. 1, 2022
(86) PCT No.: PCT/US2022/075841

§ 371 Date: Mar. 21, 2024

Related U.S. Application Data
(60) Provisional application No. 63/248,768, filed on Sep. 27, 2021.

(51) Int. Cl. A61K 47/68; A61K 47/69; C07K 16/28

(52) U.S. Cl. CPC A61K 47/6898; A61K 47/6901; A61K 47/6903; C07K 16/2809; C07K 16/2818; C07K 2317/74

(57) ABSTRACT

Disclosed herein are cell-targeting complexes that are coated on the surface with target specific antibodies for induction of biological stimulus in target cells/tissue/organs. In some embodiments, the cell-targeting complex involves nonnucleated (e.g. platelets, red blood cells (RBC)) or enucleated cells that have been thiolated, streptavidinylated, and then coated with biotinylated antibodies. In some embodiments, the cell-targeting complex involves multilayer alginate hydrogel beads that have been coated with polyanionic proteins using a polycation, which is then thiolated, streptavidinylated, and then coated with biotinylated antibodies.

### CROSS-REFERENCE TO RELATED APPLICATIONS

This application claims benefit of U.S. Provisional Application No. 63/248,768, filed Sep. 27, 2021, which is hereby incorporated herein by reference in its entirety.

### BACKGROUND

Over the past few decades, numerous studies have elucidated the molecular and cellular characteristics of T cells that have led to new strategies to fight against this battle such as checkpoint blockade, adoptive cellular therapy etc. Adoptive cellular therapy is a procedure where lymphocytes are differentiated and/or genetically modified and expanded ex vivo and then reinfused back into the body to enhance the therapy against cancer, autoimmune disease, and viral infections. To facilitate these studies, it is crucial to have robust platforms for ex vivo differentiation and/or genetic modification, and expansion and maintenance of T cells.

Commonly used approach for T cell expansion is the use of iron-dextran nanoparticles or super-paramagnetic polymer beads coated with optimized mixture of monoclonal antibodies against the CD3 and CD28 cell surface molecules of T cells, therefore serving as artificial antigen presenting cells (APCs). These strategies have been used as a convenient reagent for expansion of T cells and to enhance the anti-tumor effect of donor lymphocyte infusions after transplantation. Over the decades studies have established that these beads can be used to expand functional T cells, and that some of these cells can persist in vivo post infusion. One of the major disadvantage of using this approach is the immobilization of antibodies on to an artificial solid a rigid surface. This results in continuous stimulus leading to exhaustion of cells activity over the time and in few cases loss of activity in vivo and thus failure of exorbitantly expensive therapy.

### SUMMARY

Disclosed herein are cell-targeting complexes involving non-nucleated or nucleated cells having cell surface proteins that have been thiolated and coated with a streptavidin, avidin, or a derivative or analogue thereof (generally referred to herein as “streptavidinylated”) with high affinity for biotin, using a sulfo-SMCC linker, and then coated with one or more biotinylated antibodies.

In some embodiments, the cell-targeting complex involves non-nucleated (e.g. platelets, red blood cells (RBC)) or enucleated cells that have been streptavidinylated by the methods disclosed herein and then coated with biotinylated antibodies. The non-nucleated or enucleated cells can be from any suitable source, including autologous and allogeneic sources. In some embodiments, the cells are RBCs from a compatible donor. For example, in some embodiments, the RBCs are O-neg or O-pos.

There are several advantages of using non-nucleated or enucleated cells in place of rigid polymer beads. First, immobilization of antibodies on bilipid membrane very similar to that of natural antigen presenting cells. Over the time if the interaction between antigen-antibody is strong then target cells (e.g. T cells) can pinch the singling moiety as it happen in natural process. In addition, RBCs are known to carry and deliver oxygen to target tissues and cells, during activation phase cells/tissues need quickly a large amount of oxygen. This activation doesn't need external cytokines like IL-2. Also, RBC intracellular space can be utilized to carry different molecules to target sites.

Also disclosed is a cell-targeting complex involving multilayer alginate hydrogel beads having a cell surface that has been coated with a polyanionic protein using a polycation selected from the group consisting of as poly-D-lysine (PDL), poly-L-lysine (PLL), poly-L-ornithine (PLO), and any combination thereof, wherein the coated polyanionic protein is thiolated and streptavidinylated with a sulfo-SMCC linker, and then coated with one or more biotinylated antibodies.

In some embodiments, the polyanionic protein is selected from the group consisting of collagen, gelatin, laminin, fibronectin, or any combination thereof.

In some embodiments, the one or more biotinylated antibodies comprise anti-CD3 and anti-CD28 antibodies. In some embodiments, the one or more biotinylated antibodies bind tumor antigens.

In some embodiments, the cell-targeting complex is loaded with cytokines and/or growth factors configured to enhance activation and proliferation of T cells. In some embodiments, the cell-targeting complex is loaded with costimulatory molecules, death receptors, chemokines, and/or cytokines configured to kill cancer cells.

Target specific complexes can scan and target specific organisms/cells/tissue/organ based on its antigenicity. Following its interaction with target cells, the complexes can release factors that will stimulate a biological effect, such as induce proliferation, differentiation, activation, or cell death.

The disclosed complexes can be used in vivo, ex vivo, in vitro, or in situ. In some embodiments, the disclosed complexes are used in vivo to target cells of interest. For example, the disclosed complexes can be used to deliver a signal (e.g. activation, proliferation, differentiation, death) to any cell type based on the choice of target specific antibodies.

In some embodiments, the disclosed complexes are used in a cell manufacturing process. For example, in some embodiments, the disclosed complexes can be used in the place of feeder cells, e.g. in the cell expansions of NK cells, iPSCs, MSCs, dendritic cells, etc.

In some embodiments, the disclosed complexes are used to transport artificial oxygen carrier such as perfluorocarbon, HEMOGLOBIN-BASED OXYGEN CARRIERS, X80, FC75, FC43, FC17 (perfluorotetrahydrofuran, perfluorotributylamine), perfluorodecaline, or perfluoromethyl decaline.

With the disclosed approach, T cells can be activated and expanded by using the disclosed cell-targeting complexes mimicking antigen-presenting cells. RBCs, for example, serve as excellent targets for biomimicry due to the less complex nature (eg. lack of cell nucleus). Although biologically simple, RBCs possess unique features for biomimicry such as biconcave discoidal shape that provides optimized surface area to volume ratio and allows RBCs to undergo deformation which allows passage through vasculature. They also possess the ability to deliver oxygen through fast formation of oxyhemoglobin complexes, and the presence of biomarkers such as CD47, prevents from phagocytosis by macrophages allowing long circulation times. Due to the high biocompatibility and biodegradability, RBCs serve as excellent vehicles to deliver antigens to T cells for its activation and expansion in the absence of exogenous cytokines. Also, when using RBCs to activate and expand T cells, RBC surface is modified chemically to attach antibodies. Another advantage of this approach is the use of natural bilipid membrane layer for antibody coating, thus mimicking APCs.

In some embodiments, the complexes are prepared by the attachment of Streptavidin molecules on the surface of the non-nucleated or enucleated cells followed by the addition of biotinylated antibodies. In some embodiments, this streptavidinylation process involves the chemical modification or thiolation of surface proteins on the cells using a chemical reagent which will convert surface amines to thiols. This chemical reagent is a cyclic thioimidate compound for sulfhydryl addition. In some embodiments, Traut's reagent (2-Iminothiolane) is used for sulfhydryl addition on the cells. It reacts spontaneously and efficiently with primary amines at pH 7-9, introducing sulfhydryl groups while maintaining charge properties similar to the original amino group. The resulting surface thiols now can be linked to Streptavidin molecules using a linker, sulfo-SMCC, a heterobifunctional crosslinker that contains N-hydroxysuccinimide (NHS) ester and maleimide groups that allow covalent conjugation of amines in streptavidin molecules and sulfhydryls in thiolated RBCs. Once streptavidin is attached on to the cell surface, biotinylated antibodies can be added to prepare the complexes.

In some embodiments, the number of biotinylated antibodies on each complex in a mixture of cell-targeting complexes is within 10, 20, 30, 40, or 50% of the mean.

In some embodiments, each complex in a mixture of cell-targeting complexes are coated with 50,000 to 1,000,000 biotinylated antibodies, including 100,000 to 500,000 biotinylated antibodies.

In some embodiments, the complexes are prepared by modifying the cells to express streptavidin or a species specific Fc receptor.

In some embodiments, the complex is configured to deliver one or more signals to T lymphocytes (T Cells) in a mixture of peripheral blood monocyte cells (PBMC). The complex can be designed to activate and expand human T cells from mixture of human PBMC using the disclosed cell-targeting complexes mimicking antigen-presenting cells. Due to the high biocompatibility and biodegradability RBCs serve as excellent vehicle to deliver antigens to T cells for its activation and expansion in the absence of exogenous cytokines.

In some embodiments, the complex is loaded to release factors that will stimulate a biological effect, such as induce proliferation, differentiation, activation, or cell death.

In some embodiments, the complex is coated with tumor specific antibodies and loaded with costimulatory molecules, death receptors, chemokines, and/or cytokines to target and kill cancer cells.

Also disclosed herein is a method for expanding, differentiating, or killing a cell in a culture system, that involves culturing the cell in the presence of a cell-targeting complex disclosed herein. In some embodiments, the cell-targeting complex is configured as a feeder layer for the cell.

For example, targeted killing can involve in some embodiments, the cells (e.g. RBC) are surface labelled with death inducing ligand such as TNF-alpha, FasL (Apo-2L), TRAIL (Apo-2L), Apo-3L (Tweak) and/or antibodies to death receptors on target cells and/or reagents (antibodies) to deprive target specific growth factors (trophic factors). In some embodiments, the cells (e.g. RBC) are loaded with cytotoxic agents such as granzyme, perforin, tunicamycin, 6-OHDA, Campothecin, etoposide, cytotoxic drugs (cisplatin, taxol, mitoxantrone, idarubicin, Adriamycin etc.), saturosporin to kill the cells of interest.

For targeted differentiation, the cells (e.g. RBC) can be surfaced labelled with target antigen specific antibodies/peptide/ligand and also with cytokines IL-4 for Th2 differentiation, INF-gamma & IL-12 for Th1 differentiation, TGF-beta, IL-6 and IL-21 for TH17 differentiation, IL-6 and IL-21 for T-follicular helper cells (Tfh).

In some embodiments, differentiation of stem cells to islet cells can involve cells (e.g. RBCs) coated with stem cell targeting antibodies/peptide/ligand and biotinylated-Activin-A, and subsequent addition of following biotinylated recombinant proteins or RBCs tagged with following proteins—RA, FGF-10, Noggin, Exendin-4, Betacelluline and Wnt-4.

In some embodiments, culturing of embryonic stem(ES) cells on streptavidinylated cells (e.g. RBCs) coated with Leukemia Inhibitory Factor (LIF) followed by subsequent addition of bioninylated-transferrin and—noggin or SA-RBC coated with transferrin and noggin, following 3-5 day of cultures these cells can be further differentiated using addition of biotinylated-bFGF, -FGF-8, -Noggin, and -Laminin for additional 6-8 days to get neuronal precursors these cells can be further subdivided using SA-RBC coated with biotinylated-growth factors.

In some embodiments, to produce dendritic cells, bone marrow derived precursors or peripheral monocytes can be cultured with streptavidinylated cells (e.g. RBCs) coated with biotinylated-IL-4 and-GMCSF for 3-6 days to get immature DC. The immature DC can be further differentiated using SA-RBC conjugated with biotinylated-GMCSF.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 illustrates an embodiment of a disclosed complex having the ability to carry multifunctional therapeutic agents which will scan, target, and kill cancerous cells. These beads can be loaded with tumor screening devices (tumor specific antibodies), costimulatory molecules, death receptors, chemokines, and/or cytokines.

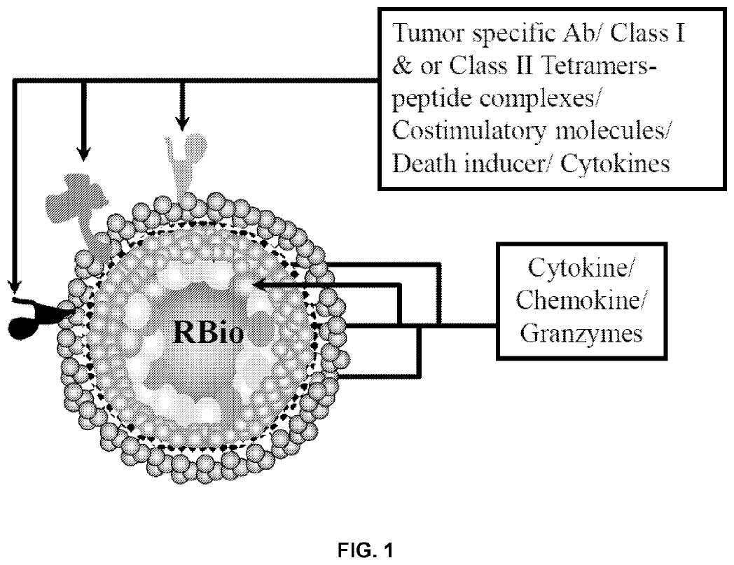

FIG. 2 shows an example protocol for conjugating antibodies onto the surface of RBCs.

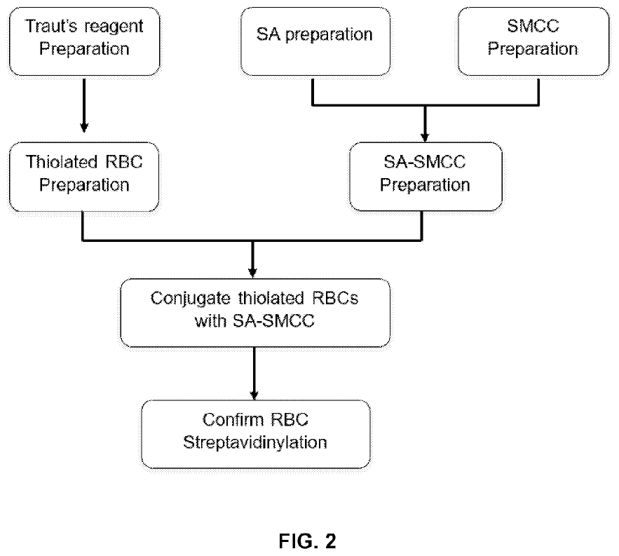

FIG. 3 illustrates an embodiment of a disclosed RBC or alginate bead coated with antibodies that specifically bind and activate cells, such as T-cells, B-cells, NK cells, Dendritic cells, stem cells, or iPSCs.

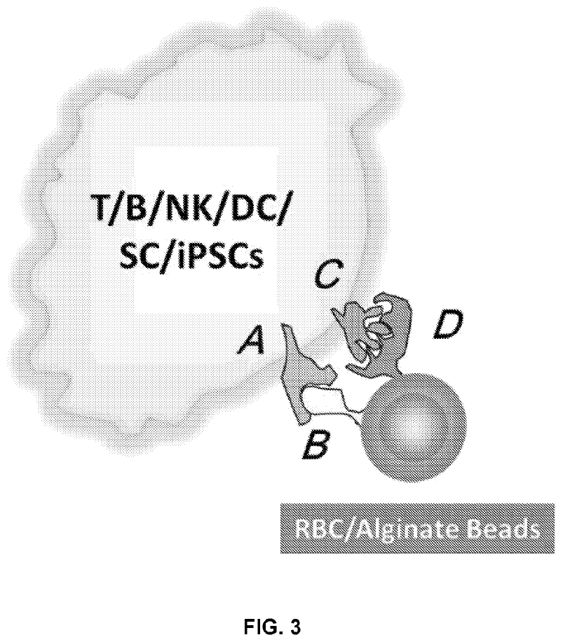

FIG. 4 illustrates an embodiment of a disclosed bead coated with antibodies that specifically bind CD28 and CD3 to activate T-cells.

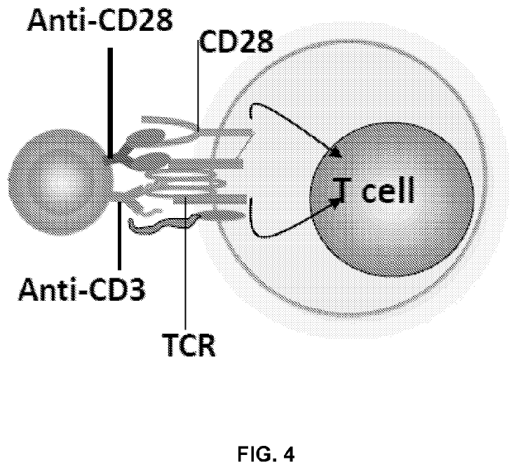

FIG. 5 shows activation of CD4 T cells using the beads disclosed herein.

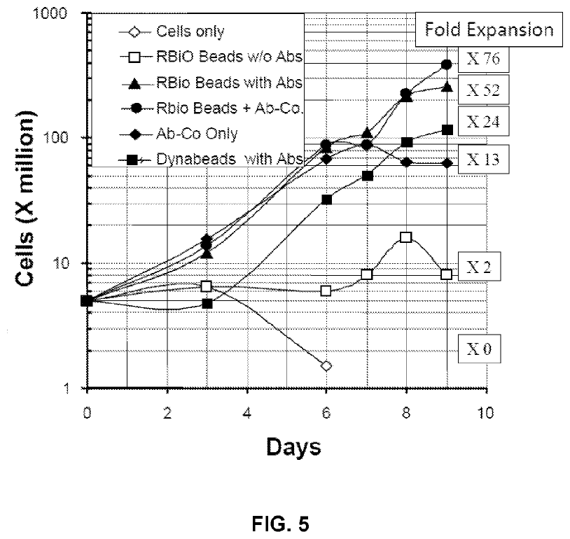

FIG. 6 illustrates a strategy for NK cell activation and proliferation using beads disclosed herein coated with antibodies that specifically bind NKp30, CD3, NKG2D, FCR, and/or IL-15R.

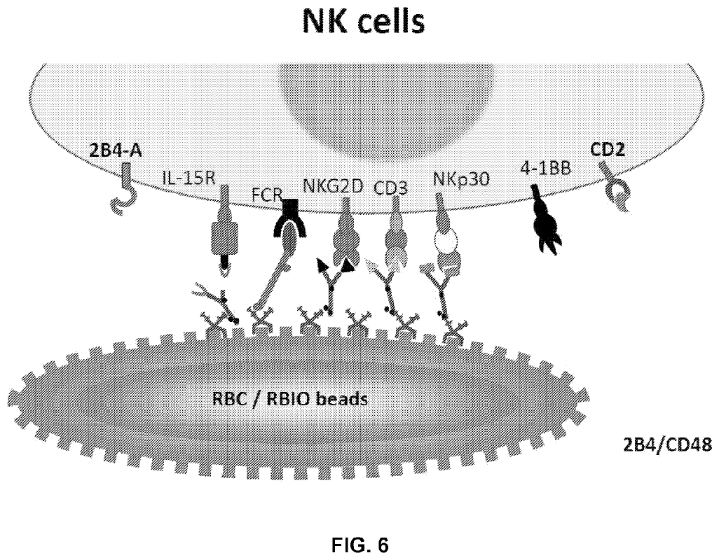

FIGS. 7A and 7B show assessment of Streptavidinylated-RBC (SA-RBC) conjugation. Shown is flow cytometry analysis of FITC stained RBCs conjugated with Streptavidin. Shown are unlabeled RBCs (mean fluorescence intensity (MFI); 4.28) and FITC labelled SA-RBC conjugates (MFI; 648). FIG. 7A shows a dot plot representation FIG. 7B is a histogram representation of data. As shown in the graphs FITC RBC-Streptavidin conjugates (RBC-SA) conjugates has about >100-fold MFI compared to the unstained control.

FIG. 8 shows stability assessment of RBCs, Thiolated-RBC (RBC_A) and RBC-SA (RBC-B conjugates). Shown is the percent Lysis of RBC, RBC_A and RBC-B conjugates stored in RB RBC storage buffer at 2×10⁹ cells/mL cell density up to 2 weeks at 4° C. As depicted in the figure <2.5% lysis was observed when RBCs are stored at the conditions mentioned above up to 2 weeks' time.

FIG. 9 shows stability assessment of RBC-SA (RBC-B) conjugates. Shown in s the mean fluorescence intensity of RBC-SA conjugates stored in RB RBC storage buffer at 2×10⁹ cells/mL cell density up to 9 Months. Statistical analysis was performed using ordinary one-way ANOVA followed by Tukey's post hoc test for multiple comparisons. P<0.05 was considered as significant. There was no statistically significant difference between samples at each time point. As depicted in the figure, when RBC-SA conjugates are stored at conditions mentioned above, they are stable up to 9 Months.

FIG. 10 shows activation/proliferation of macrophages depleted human PBMC using hu-anti-CD3 and hu-anti-CD28 conjugated to GAM beads (Dynabeads) vs RBC. Cell growth is shown as Log 2 cell number (×10⁶) over the 14 days of post activation.

FIG. 11 shows activation/proliferation of human Pan-T cells using hu-anti-CD3 and hu-anti-CD28 conjugated to GAM beads (Dynabeads) vs RBC. Cell growth is shown as Log 2 cell number (×10⁶) over the 14 days of post activation.

FIG. 12 shows activation/proliferation of NK cells in PBMC cultures using streptavidinylated RBCs coated with hu-anti-CD3, hu-anti-NKG2D and IL-15 plus soluble ILD2 and irradiated K562 cells. Plot depicting human PBMC (NK) growth as Log 2 cell number (×10⁶) versus days post activation up to 14 days.

FIG. 13 shows representative immunofluorescence (IF) staining of alginate-PLO (AL-PLO) microbeads. Panels A, C and E are stained with isotype control antibody conjugated with FITC; Panels B, D and F are stained with anti-streptavidin (a-SA) antibody conjugated with FITC. Panels A & B are non-reduced AL-PLO microbeads; Panels C & D are AL-PLO microbeads reduced by Traut's reagent; Panels E & F are streptavidinylated-AL-PLO microbeads. Immunofluorescent images were captured at 4× magnification on the Lionheart™ FX Automated Microscope (BioTek Instruments Inc., Winooski, VT).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “target cell directed moiety” refers to a moiety that interacts with a receptor on a target cell. The target cell directed moiety can be an entire molecule or a portion of a molecule. The target cell directed moiety can be included within or attached to another molecule as long as the target cell directed moiety is capable of interacting with the receptor on the target cell.

The term “target cell receptor” refers to a molecule on the surface of a target cell that, upon interaction with a target cell directed moiety/RBC complex, participates in and/or contributes to the stimulation a of biological effect in the target cell.

The term “red blood cells” includes hemoglobin-containing erythrocytes, erythroblasts and reticulocytes, as well as hemoglobin-depleted red blood cell “ghosts.”

The term “T cells” refers to CD4-positive or CD8-positive lymphocytes that express the CD3 antigen.

The term “activated T cells” refers to T cells that have undergone differentiation to a particular subset of T cell. Activated T cells include, but are not limited to, Th1 and Th2 subsets. Activated T cells include any T cell subtype and are not limited to any particular defined cytokine profile. As used herein, activated T cells may refer to either polyclonal or monoclonal populations of T cells.

The term “antibody” refers to a polypeptide or group of polypeptides which are comprised of at least one antibody combining site. An “antibody combining site” or “binding domain” is formed from the folding of variable domains of an antibody molecule(s) to form three-dimensional binding spaces with an internal surface shape and charge distribution complementary to the features of an epitope of an antigen, which allows an immunological reaction with the antigen. An antibody combining site may be formed from a heavy and/or a light chain domain (VH and VL, respectively), which form hypervariable loops which contribute to antigen binding. The term “antibody” includes, for example, vertebrate antibodies, hybrid antibodies, chimeric antibodies, altered antibodies, univalent antibodies, the Fab proteins, and single domain antibodies.

### Non-Nucleated or Enucleated Cells

Disclosed herein are complexes comprising non-nucleated (e.g. platelets, red blood cells (RBC)) or enucleated cells coated with target cell directed moieties as well as methods of using these coated RBCs or platelets to stimulate a biological effect in target cells. When the complex contacts the target cell, the moiety interacts with a receptor on the target cell and the interaction stimulates a biological effect in the target cell.

The use of complexes comprising RBCs for stimulation of a biological effect in target cells is disclosed in WO2004087876, which is incorporated by reference herein for the teaching of these complexes. As disclosed therein, presenting a target cell directed moiety on the surface of an erythrocyte or red blood cell (RBC) to the target cell is particularly effective in stimulating a biological effect in the target cell. The target cell directed moiety interacts with a receptor on the target cell and a biological effect is stimulated in the target cell. Disclosed herein are improved methods for coating the moiety on the surface of an RBC or platelet.

The use of an RBC or platelet as a component of a target cell stimulating complex offers distinct benefits for and advantages to stimulating a target cell. Presentation of a target cell directed moiety on the surface of an RBC or platelet generally provides a local concentration of the moiety to the target cell through the presence of a number of moieties on the surface of an RBC or platelet. In some instances, coupling of the target cell directed moiety to the surface of an RBC or platelet allows for some mobility of the moiety when interacting with the target cell receptor and, accordingly, for mobility and/or aggregation of the target cell receptor. For some cell receptors, the ability to aggregate and/or move on the cell surface is important for effective cell signaling. In some instances, the RBC or platelet complexes can also be used to deliver agents (e.g., drugs, antigens, cytokines, chemokines, hormones) to particular cells and/or tissues. In addition, the use of RBC or platelet in the presentation of target cell directed moieties to target cells provide a source of oxygen to the cells in culture or in the individuals to which the complexes are administered.

In some embodiments, the streptavidinylation process involves the chemical modification or thiolation of surface proteins of cells using a chemical reagent which will convert surface amines to thiols. This chemical reagent is a cyclic thioimidate compound for sulfhydryl addition. For example, Traut's reagent can be used for sulfhydryl addition on the cells. It reacts spontaneously and efficiently with primary amines at pH 7-9, introducing sulfhydryl groups while maintaining charge properties similar to the original amino group. The resulting surface thiols now can be linked to Avidin, Straptavidin, Strep-tactin, or other streptavidin mutant molecules using a linker, such as sulfo-SMCC, which is a heterobifunctional crosslinker that contains N-hydroxysuccinimide (NHS) ester and maleimide groups that allow covalent conjugation of amines in streptavidin molecules and sulfhydryls in thiolated RBCs. Once streptavidin is attached on to the RBC surface, biotinylated antibodies can be added to prepare the RBC-SA-3/28 complexes which will be used for T cell activation.

### Multilayer Alginate Hydrogel Beads

In some embodiments, the cell-targeting complex involves multilayer alginate hydrogel beads that have been streptavidinylated by the methods disclosed herein and then coated with biotinylated antibodies.

Alginate beads can be prepared using methods known in the art and disclosed herein. For example, microcapsules/microbeads can be formed using air dispersion principle where a controlled flow of filtered Ultrapure Nitrogen disperses a jet of alginate solution, along with its components, into droplets of defined size and shape which fall into a crosslinking agent bath (e.g. CaCl₂). Biological agents can be mixed into the sodium alginate solution at desired concentration prior to the encapsulation process in order to form a therapeutic product. The size of the microcapsules is precisely controlled using optimized injection flow rates and pressure of the dispersion gas. The final product can be collected in a sterile container and filtered through a size filter.

These beads can be coated proteins using a polycation, such as poly-D-lysine (PDL), poly-L-lysine (PLL), and/or poly-L-ornithine (PLO). In some embodiments, these polycations are used to coat the alginate bead with a protein that can be thiolated and then streptavidinylated as described above. Examples of suitable proteins Collagen, Gelatin, Laminin, Fibronectin, or any combination thereof.

The alginate and or hyaluronic acid beads can be resuspended in appropriate concentration of coating agents (e.g. between 0.00001 mg/ml to 10 mg/ml) in a continuous mixing device for 30 min to 24 hrs as per desire coating thickness. Following the incubation period excess coating reagent can be removed by decanting and the coated beads washed. These multilayered beads can then subjected to the thiolation and streptavidinylation process described above for cells.

In some embodiments the disclosed methods of preparation of microcapsules/microbeads alginate can be replace with hyaluronic acid, hyaluronic acid, chitosan, agarose, and dextran, and proteins, such as gelatin and collagen, alone or in combination.

### Stimulating a Biological Effect in Target Cells

The degree and/or type(s) of biological effect stimulated in a target cell depends on a number of factors, including, for example, the target cell type, the target cell receptor, the target cell directed moiety and the presentation of the moiety to the target cell receptor. In some cases, interaction of a target cell directed moiety alone with a target cell receptor may stimulate a biological effect in the target cell. In other cases, presentation of the target cell directed moiety on the surface of the disclosed cell-targeting complexes is necessary to stimulate a measurable biological effect in the target cell. In either case, presentation of a target cell directed moiety coupled to the surface of disclosed cell-targeting complexes to the target cell receptor is an effective way to stimulate a biological effect in the target cell. As exemplified herein, contacting a target cell with a target cell directed moiety coupled to a linker coupled to disclosed cell-targeting complexes is more effective in stimulating a biological response than contacting the target cell with the target cell directed moiety alone or with the target cell directed moiety coupled to a bead.

In the disclosed methods, stimulation of a biological effect in the target cell results from contacting the target cell with at least one target cell directed moiety attached to the surface of disclosed cell-targeting complexes. In some embodiments, stimulation of a biological effect in the target cell requires interaction of more than one target cell directed moieties coupled to disclosed cell-targeting complexes with one or more target cell receptors. In some embodiments, stimulation of a biological effect in the target cell requires interaction of more than two target cell directed moieties coupled to disclosed cell-targeting complexes with one or more target cells receptors.

Examples of target cells include, but are not limited to, cells of the immune system, bone marrow cells, stem cells, infected cells, hyperplastic cells and tumor and/or cancer cells. Exemplary target cells include T cells, natural killer (NK) cells, tumor infiltrating lymphocytes (TIL), lymphokine-activated killer (LAK) cells, B cells, monocytes, granulocytes, macrophages, immature and mature dendritic cells, Induced Pluripotent Stem Cells (iPSCs) and mesenchymal stem cells (MSCs). The target cell may also be any non-cancerous cell that could provide a direct or indirect therapeutic response.

Examples of biological responses that can be stimulated in cells of the immune system, bone marrow cells, and/or stem cells, include, but are not limited to, activation, proliferation, differentiation, and/or induction of cytokine production. Examples of biological responses that can be stimulated in non-immune system cells include, but are not limited to, production of hormones, neurotransmitters and/or other biological response molecules. Examples of target cell directed moieties that can stimulate such biological effects in such cells are listed herein.

Examples of biological responses that can be stimulated in infected cells, hyperplastic cells, tumor cells and/or cancer cells include, but are not limited to, anti-proliferative responses, cytotoxic effects, apoptosis, and necrosis. Examples of target cell directed moieties that can stimulate such biological effects in such cells are listed herein.

In some embodiments, the disclosed method is appropriate for use in vitro and/or in vivo. For example, target cells in culture can be contacted with the complexes according to the methods and, once the biological effect is stimulated, the cells and/or culture media can be harvested for further use. In some embodiments, methods of the invention can be used for ex vivo purposes, for example, where cells are collected from an individual and put in culture conditions as needed, the biological effect is stimulated according to the methods of the invention and the resultant cells and/or cell products, are administered to an individual in need thereof.

For example, target cells can be contacted in culture with the disclosed cell-targeting complexes to stimulate an increased level of production and/or secretion of a variety of cytokines. The cytokine(s) in the cell culture supernatant can be separated from the target cells and disclosed cell-targeting complexes and used for a variety of purposes including administration to a subject in need thereof. The disclosed cell-targeting complexes can be used to stimulate cytokine production from a homogeneous cell population (e.g., a population enriched for a particular subset of cells, e.g., CD4+ T cells) or from a heterogeneous cell population. The particular cytokine(s) stimulated by the target cell directed moiety/complexes depends on the target cell population and on the target cell directed moiety used for the stimulation. For example, cells from the immune system can be stimulated to produce cytokines including, but not limited to, IL-2, IL-4, IL-5, IL-6, IL-8, IL-10, IL-12, IL-15, IL-17, IL-18, IL-21, IL-27, IL-35, TRAIL, FasL, IFN-γ, TNF-α, BAFF, APRIL, LT-α and TNF-β. Target cell directed moieties for stimulation of cytokine production include those described herein, such as a lectin (e.g., PHA) or an anti-target cell receptor antibody (e.g., anti-CD3 and anti-CD28 antibodies).

In stimulating cytokine production or secretion from the cells in culture, the target cell directed moiety/complexes may be added to the cells once or repeatedly. Separation of the cytokine-containing culture supernatant from the cells and complexes can be done using separation technologies including filtration, precipitation, fractionation and sedimentation. In some embodiments, the culture supernatant containing the desired cytokine(s) is removed from the cells and complexes prior to substantial cell lysis and without causing substantial cell lysis. since the surface of the disclosed cell-targeting complexes is streptavidinylated (i.e., coupled with streptavidin), biotinylated magnetic particles and an application of a magnetic field can be used to remove the complexes and any cells attached to the complexes from the culture supernatant. This can be accomplished in a batch mode (e.g., using a permanent magnet) or in a continuous mode by flowing the mixture of complexes, target cells and cell culture supernatant over a permanent magnet. Where contact with the target cell directed moiety/complex causes the target cells to proliferate, the target cell culture can be saturated by the addition of excess complex prior to removal of the cells and complexes from the supernatant. Cytokines of the cell culture supernatant can be further purified using techniques known in the art, including, for example, using cytokine-specific affinity columns.

In another example of ex vivo stimulation, T cells can be isolated from peripheral blood mononuclear cells (PBMCs) and stimulated to proliferate and/or differentiate into, for example, Th1, Th2, Th0, Tc1, Tc2, Th9, Th17, Th22, Treg/Th3 or any activated T cell subtype not limited to a particular defined cytokine producing profile. As exemplified herein, T cells can be isolated from PBMCs and stimulated to proliferate and differentiate into activated T cells according to a method of the invention. The activated T cells so generated can then be administered to individuals, for example, for adoptive immunotherapy.

For example, target T cells can include those of any antigen specificity, including non-antigen specific, and include T cell populations that are monoclonal or polyclonal. T cells that result from the methods of the invention include those of any antigen specificity, including non-antigen specific, and monoclonal or polyclonal T cell populations. Also, methods of the invention can be used to generate T cells of any effector profile including any surface marker profile or any cytokine profile.

In the methods described herein, the target cells can be stimulated with the target cell directed moiety/complex once or repeatedly until the desired effect is obtained. In some embodiments, following stimulation of the target cells with the target cell directed moiety/RBC complex, the cells can be stimulated with other agents that serve to further result in the desired effect. As demonstrated herein, stimulation of CD4+ T cells first with anti-CD3/anti-CD28/RBC complexes and subsequently with anti-CD3/anti-CD28/streptavidin complexes (SA-CD3/CD28) resulted in effective expansion and differentiation of the population of T cells. Thus, in some embodiments, the methods further comprise contacting the target cells with target cell directed moiety/SA complexes, once or repeatedly.

In some embodiments, the disclosed methods are performed in vivo. In such methods, the target cell is contacted after the target cell directed moiety/complex(es) is administered to an individual. The administered complexes contact the target cell and stimulate a biological effect in the individual. Many of the complexes described herein are appropriate for use in vivo, including, but not limited to, those that are particularly selective for the target cell and that stimulate target cell growth arrest or apoptosis, that stimulate target cell proliferation and that stimulate target cell differentiation.

In some embodiments, methods are provided for modulating immune system function. The complexes and/or compositions of the invention are administered to subjects in need of immune system modulation in amounts effective to modulate immune system function. Modulation of immune system function includes, but is not limited to, increasing immune function such as by specifically stimulating T cells (including cytotoxic T lymphocytes (CTL)), B cells, NK cells, bone marrow cells, monocytes, macrophage, immature dendritic cells, mature dendritic cells, stem cells and/or early lineage progenitor cells to produce a prophylactic or therapeutic result relating to infectious disease, cancer, and the like. Specifically included is the use of particular complexes of the invention for the treatment of disorders characterized by reduced T cell levels in vivo, e.g., HIV and other disorders associated with a compromised immune system. Modulation of immune system function also includes, but is not limited to, decreasing immune function such as by suppressing specifically the immune system to treat autoimmune disease, allergy and the like. In one embodiment, the complexes of the invention are used to shift a Th2-type immune response toward a Th1-type immune response through the stimulation of Th1 cell production, as described herein. In another embodiment, the disclosed compositions are used to stimulate blood cell proliferation and/or differentiation.

Also disclosed are methods and compositions for increasing the size of a subpopulation of T cells. In such methods, “increasing size of a subpopulation of T cells” refers to stimulating the expansion of a T cell subpopulation by contacting the T cells with at least one complex comprising a T cell directed moiety coupled to a disclosed cell-targeting complex where the interaction of the T cell directed moiety with a T cell receptor stimulates proliferation or expansion of the T cell subpopulation of cells. In some embodiments, the number of T cells belonging to the subpopulation that are present after this contacting is at least 2, 3, 4, 5, 10, 15, 20, 30, 40, 50, 70, 90, 150, 500, 5000, 50,000 or 100,000 fold greater than the number of these cells present without administration of the complexes or after the corresponding control incubation in the absence of the complexes. In some embodiments, the number of T cells belonging to the subpopulation that are present after this contacting is at least 2, 3, 4, 5, 10, 15, 20, 50, 100, 1000, 10,000 or 100,000 fold greater than the number of these cells present after the corresponding in vivo contact or in vitro incubation in the presence of the same T cell directed moiety attached to the surface of a bead. It is also contemplated that the percentage may remain the same but the actual numbers of the relevant subset may increase if the total number of T cells increases. In some instances, the change in the percentage of cells that belong to the subpopulation of T cells is at least 2, 3, 4, 5, 10, 20, 50, 100, 1000, 10,000 or 100,000 fold greater than corresponding change in the percentage of cells that belong to the subpopulation of T cells in absence of administration of the complexes, in a control sample that has not be incubated with the complexes or after the corresponding incubation in the presence of the same T cell directed moiety attached to the surface of a bead.

For example, methods are provided for stimulating production of Th1, Th2, Th9, Th17, Th22 or T_{FH} cells, subsets of T helper cells. The Th1 subset is responsible for classical cell-mediated functions such as delayed-type hypersensitivity and activation of cytotoxic T lymphocytes (CTLs). Thus, the Th1 subset may be particularly suited to respond to viral infections, intracellular pathogens, and tumor cells because it secretes IFN-γ and other cytokines, which activate other components of the immune system, such as CTLs. The Th2 subset suppresses the cellular immune response and functions more effectively as a helper for B-cell activation and eosinophilic inflammation. The Th2 subset may be more suited to respond to free-living bacteria and helminthic parasites and may mediate allergic reactions, since IL-4 and IL-5 are known to induce IgE production and eosinophil activation, respectively.

Differences in the cytokines secreted by Th1 and Th2 cells are believed to reflect different biological functions of these two subsets. In general, since distinct patterns of cytokines are secreted by Th1 and Th2 cells, one type of response can moderate the activity of the other type of response. A shift in the Th1/Th2 balance can result in an allergic response, for example, or, alternatively, in an increased CTL response. The disclosed methods can also be used to redirect a Th2 immune response.

Also disclosed are methods for producing a population of Th1 cells from a blood sample in the absence of exogenous growth or differentiation factors, such as IL-2 or IFN-gamma. Mononuclear cells collected from a blood sample, for example, by leukapheresis, serve as a source material for production of Th1 cells in culture. In an exemplary embodiment, CD4+ T cells are first purified from the source material. Such a purification can be accomplished by, for example, positive selection. The starting population of T cells are then contacted with the complexes of the invention to stimulate the desired biological effect in the T cells. In one embodiment, the cells are activated by simultaneous contact with a first moiety/RBC complex that interacts with the CD3 receptor complex on the T cells and a second moiety/RBC complex which interacts with the CD28 receptor on the T cells. In an exemplary embodiment, the activation is accomplished by co-incubating the starting population of T cells with anti-CD3 antibodies coupled to the surface of a disclosed cell-targeting complex and anti-CD28 antibodies coupled to the surface of a disclosed cell-targeting complex. In these embodiments, the anti-CD3 and/or anti-CD28 antibodies may be directly or indirectly coupled to the surface of the disclosed cell-targeting complexes.

The T cells are stimulated with the T cell directed complexes one or more times, typically two or more, three or more, four or more, five or more times. In an exemplary embodiment, the T cells are stimulated three times with the anti-CD3/anti-CD28/RBC complexes over the course of 9 days in culture. The repeated stimulation of the cells resulted in an expansion of T cell number in excess of 52-and 217-fold, depending on the particular preparation of complexes used. Stimulation with the antibodies alone resulted in T cell expansion of about 13-fold. In another embodiment demonstrated herein, T cells are stimulated first with anti-CD3/anti-CD28/RBC complexes and subsequently with anti-CD3/anti-CD28/streptavidin complexes (SA-CD3/CD28) over the course of 10 days in culture. The repeated stimulation with these two types of complexes resulted in an expansion of T cell number of 32-118-fold.

Cells resulting from the exemplified expansions have a Th1 phenotype as demonstrated by their production of IFN-gamma, their lack of production of IL-4 and their cell surface markers. Thus, the invention provides methods for increasing the size of a subpopulation of activated T cells. Methods are also provided for producing large numbers of activated T cells.

Activated T cells, such as Th1 cells, would be of use in treating symptoms of individuals with cancers, infectious diseases, allergic diseases and diseases or disorders that are associated with overactive humoral immunity. Individuals with cancer and tumor-bearing animals have been shown to exhibit suppressed cellular immune responses as evidenced by decreased DTH, CTL function and NK activity (Broder et al. (1978) N. Engl. J. Med. 299:1335-1341) apparently due to a lack of Th1 cells. Excess production of Th2 cytokines and/or depressed production of Th1 cytokines resulting in a Th1/Th2 cytokine imbalance has also been reported in virtually all types of cancer tested. As with asthma and allergies, enhanced Th2 responses are found in a variety of infectious diseases, such as chronic hepatitis C virus infection (Fan et al. (1998) Mediators Inflamm. 7:295), leprosy (Yamamura (1992) Science 255:12), toxoplasmosis (Sher et al. (1992) Immunol. Rev. 127:183) and AIDS (Clerici et al. (1993) Immunol. Today 14:107-111), and autoimmune conditions, such as lupus (Funauchi et al. (1998) Scand. J. Rheumatol. 27:219).

Therapies that increase antigen specific Th1 cells and/or shift the balance from Th2 to Th1 have been shown to have therapeutic utility in treating cancer and infection conditions. For example, down-regulation of the Th2 response in tumor-bearing mice by treatment with anti-IL-4 mAb significantly suppresses growth of murine renal cell carcinoma tumors (Takeuchi et al. (1997) Cancer Immunol. Immunother. 43:375-381), while IL-2 gene transfected murine renal cell carcinoma cells mediate tumor rejection (Hara et al. (1996) Jpn. J. Cancer Res. 87:724-729). IL-2 is a Th1 associated cytokine. Adoptive immunotherapy involving transfer of influenza-specific Th1 cells was protective against influenza infection, while Th2 infusion failed to induce protection (Graham et al. (1994) J. Exp. Med. 180:1273).

Th1 cells that can be used in adoptive immunotherapy for a variety of conditions in which an increase in the population of Th1 cells would be of beneficial, such as in treatment of a variety of diseases, including cancer, infectious disease, allergy and diseases characterized by overactive humoral immunity, such as systemic lupus erythematosus. Methods of the invention in which complexes that stimulate differentiation of T cells to Th1 cells are administered can be used to shift a Th2 immune response toward a Th1 immune response in an individual in need thereof. Methods of the invention can also be used to stimulate production of Th1 cells in an individual in need thereof.

In some embodiments, the disclosed methods involve the use of disclosed cell-targeting complexes as artificial antigen presenting cells (APCs) to stimulate T cells to respond a particular antigen, such as a tumor antigen or an antigen associated with infectious disease. RBC or platelet compositions that can be used as artificial APCs include those which have a specific antigen, or fragment thereof, coupled to the RBC or platelet cell surface. Artificial APCs may also include compositions comprising RBCs or platelets having cell surface coupled major histocompatability complex (MHC) molecules (such as, class I or class II molecules) loaded with antigen peptide. Such compositions may be used, for example, in methods to prime T cells in vitro.

In some embodiments, the disclosed methods involve the use of disclosed cell-targeting complexes to deliver antigens to cells or to a subject in need thereof, in particular, to deliver antigen to particular cells and/or organs of the immune system, such as lymph nodes and spleen. In these methods, the antigen of interest can be concentrated within the disclosed cell-targeting complexes and administered parenterally, such as by intravenous delivery. For example, RBCs or platelets can be collected by the spleen and the antigen can thus be delivered to the T and B cells of the spleen. Also, the disclosed cell-targeting complexes can be directed to a particular site through coupling a ligand to the complex surface that will preferentially direct the complex to the desired cells and/or organ. For example, coupling LFA-1 and CD62L to the surface of the complex prior to administration would result in the delivery of the antigen/complex to the lymph nodes. Antigen/complexes directed to the lymph nodes may further include an antigen linked to a Tat polypeptide of HIV or any other appropriate signaling peptide which facilitates processing of the antigen. Given the directed aspect of this form of antigen administration, the spread of antigen in the individual would be restricted to the particular desired sites and lower doses of antigen can be delivered since it is preferentially directed to sites where it would be most useful.

Also disclosed are methods for suppressing proliferation of target cells and/or for inducing cell death in target cells. The complexes and/or compositions disclosed herein can be administered to subjects in need of suppression of cell proliferation and/or induction of cell death in amounts effective to suppress target cell proliferation and/or to induce cell death in the target cell. Such individuals include those with cancer, tumor cells, infected cells and/or diseases or disorders characterized by cell proliferation. Suppressing proliferation (including, for example, through slowing or arresting cell division) and/or inducing cell death (including, for example, through stimulating apoptosis) in target cancer cells, tumor cells, and/or infected cells produces a prophylactic or therapeutic result relating to cancer, infectious disease, and the like.

In some embodiments, methods for suppressing cell proliferation involve the use of complexes with a coupled target cell directed moiety that interacts with a receptor on the target cell that stimulates suppression of proliferation and/or induction of cell death in the target cell. Such a receptor on the target cell is herein referred to as a “negative signaling” receptor. As used herein, “negative signaling” refers to the inhibition of cell growth, for example, by cell cycle arrest or the induction of apoptosis (programmed cell death).

Negative signaling receptors and their ligands are known in the art and include, for example, the tumor necrosis factor (TNF) receptor family, such as TNF receptor (TNF-R), TNF-like receptors, lymphotoxin-β receptor (LT-β-R), Fas receptor, and ligands, such as TNF, lymphotoxin-α (LT-α, formerly called TNF-β), lymphotoxin-β (LT-β), TNF-related apoptosis inducing ligand (TRAIL or Aρo-2L) and Fas ligand (FasL). TNF-R signaling is cytotoxic to cells with transformed phenotypes or to tumor cells and can lead to selective lysis of tumor cells and virus-infected cells. Like TNF-R, signaling by LT-β-R can activate pathways that lead to cytotoxicity and cell death in tumor cells. Fas receptor (Fas-R) can stimulate cytotoxicity by programmed cell death in a variety of both tumor and non-tumor cells.

The ligands TNF and LT-α bind to and activate TNF receptors p60 and p80, herein referred to as TNF-R. LT-αl/β2 heterodimeric complex binds the LT-β-R and induces cytotoxic effects on cells bearing the LT-β-R in the presence of an LT-β-R activating agent, such as IFN-gamma. See, for example, U.S. Pat. No. 6,312,691. Fas ligands are capable of inducing apoptosis in cells that express a Fas receptor. The human and mouse Fas ligand genes and cDNAs have been isolated and sequenced (Genbank Accession No. U08137).

In addition to stimulation through interaction with specific ligands, antibody binding can also activate negative signaling receptors to signal growth arrest and/or apoptosis. Antibodies that have negative signaling properties include, but are not limited to, anti-Fas, anti-LT-β-R, anti-CD40, anti-Class II MHC, anti-Her-2, anti-CD19, anti-Le^{y}, anti-idiotype, anti-IgM, anti-CD20, anti-CD21 and anti-CD22 as reported, for example, in Trauth et al. (1989) Science 245:301-305; Funakoshi et al. (1994) Blood 83:2787-2794; Bridges et al. (1987) J Immunol. 139:4242-4249; Scott et al. (1991) J. Biol. Chem. 266:14300-14305); Ghetie et al. (1992) Blood 80:2315-2320; Ghetie et al. (1994) Blood 83:1329-1336; Schreiber et al. (1992) Cancer Res. 52:3262-3266; Levy et al. (1990) J. Natl. Cancer Inst. Monographs 10:61-68; Vitetta et al. (1994) Cancer Res. 54:5301-5309; Page et al. (1988) J Immunol. 140:3717-3726; Beckwith et al. (1991) J Immunol. 147:2411-2418; U.S. Pat. Nos. 6,312,691 and 6,368,596. Furthermore, negative signaling can sometimes be optimized by hypercrosslinking with secondary antibodies or by using “cocktails” of primary antibodies (Marches et al. (1996) Therap. Immunol. 2:125-136).

In addition to the target cell directed moiety that interacts with the target cell receptor to stimulate a biological effect, the complex can also include a cell targeting molecule that directs the complex to the target cell. Such targeting molecules are components of the complex that enhance the accumulation of the complex at certain tissue or cellular sites in preference to other tissue or cellular sites when administered to an intact individual, organ or cell culture. Such a targeting moiety can be inter alia-peptide, a region of a larger peptide, an antibody specific for a target cell surface molecule or marker, or antigen binding fragment thereof, a nucleic acid, a carbohydrate, a region of a complex carbohydrate, a special lipid, or a small molecule such as a drug, hormone, or hapten, attached to any of the aforementioned molecules. Antibodies with specificity toward cell type-specific cell surface markers are known in the art and are readily prepared by methods known in the art. The complexes can be targeted to any cell type in which a stimulation of the biological effect is desired, e.g., a cell type in which proliferation is to be stimulated or a cell type in which growth arrest is to be induced.

The disclosed methods can further include delivery of an agent (e.g., a drug) to cells (e.g., in culture or in an individual) or to an individual using the disclosed cell-targeting complexes as a delivery vehicle for the agent. In this embodiment, the disclosed cell-targeting complexes are loaded with an agent that will work along with the target cell directed molecule to result in the desired effect. For example, the disclosed cell-targeting complexes with a target cell directed moiety designed to stimulate T cell proliferation coupled to its surface can be loaded with a cytokine that further stimulates T cell growth (e.g., IL-2, IL-7, IL-15, IL-18, IL-27, CXCL12). Thus, the complex provides an additional stimulatory component to support T cell proliferation. In another example, the disclosed cell-targeting complexes can be loaded with an anti-apoptosis agent and a target cell directed moiety designed to send an anti-apoptosis signal to the target cell can be coupled to the surface of the complex.

In such methods, disclosed cell-targeting complexes can be loaded with one or more agents. Agents and methods for loading agents in complexes are described elsewhere herein. The disclosed cell-targeting complexes can be loaded with such agents before, during and/or after the target cell directed moiety is coupled to the complex surface.

Disclosed herein is a complex coupled to at least one moiety that interacts with a receptor on a target cell (i.e., “a target cell directed moiety. In some embodiments, the receptor on the target cell is a molecule that, upon interaction with the target cell directed complex, stimulates or contributes to stimulation of a biological effect in the target cell.

Red blood cells (RBCs) or platelets for use in the disclosed compositions and methods include RBCs or platelets isolated, for example, from whole blood, bone marrow, fetal liver, cord blood, buffy coat suspensions, pleural and peritoneal effusion, and other tissue or fluid. The RBCs or platelets can be autologous or allogeneic relative to the target cell. When administered to an individual, the RBCs or platelets can be autologous or allogeneic to the individual.

In some embodiments, the RBCs can be intact or can be depleted of hemoglobin, i.e., ghost RBCs. Ghost RBCs can be created by depleting the RBC of hemoglobin using methods known in the art including, for example, through reverse hemolysis using hypotonic/hypertonic solutions.

In some embodiments, RBC, platelets and enucleated cells are generated from genetically altered iPSc, MSCs. Also these RBC, platelets and enucleated cells are genetically modified to express streptavidin or Fc receptor or similar moiety for tagging antibodies or proteins, RNA. DNA, PNA, etc.

The RBCs or platelets may have their membranes fixed using a variety of reagents and protocols known in the art including, for example, paraformaldehyde, gluteraldehyde, formamide and the like. Fixing of the RBC or platelet membranes can provide some rigidity to the membranes. Ghost or intact RBCs can be fixed and any fixation can occur before, during and/or after the target cell directed moiety is coupled to the RBC surface.

The choice of the target cell directed moiety and the target cell receptor to which the moiety is directed depends on the target cell and the desired biological effect to be stimulated. Target cell receptors for use in stimulation of a biological effect include, but are not limited to, CD3, CD28, CD2, MHC class I complex (including dimer, tetramer, multimer) loaded with peptide, MHC class II complex (including dimer, tetramer, multimer) loaded with peptide, T cell receptor complexes (including alpha-beta and gamma-delta), CD 16, CD45, CD25, CD27, ICOS, CD40, CD40L, CTLA-4, OX-40, OX40L, CD30, CD30L, CD137, 4-1-BBL, B7.1, B7.2, FasR, FasL, TRAIL, DR4, DR5, DR3, TNFR1, TNFR2, chemokine receptors, receptors of cytokines (e.g., IL-1, IL-2, IL-3, IL-4, IL-5, IL-6, IL-7, IL-10, IL12P35, IL12P40, IL12P70, IL-13, IL-15, IL-18, IL-23, IL-27, TNF-alpha, TNF-beta, TGF-beta, IFN-gamma, GM-CSF), common gamma chain of IL-2 receptor, and any associated components of cytokine receptors. Additional examples of target cell receptors for use in stimulation of a biological effect include TNF-R, LT-βR, Her-2, CD 19, IgM, CD20, CD21 and CD22. In some instances, target cell receptors for use in stimulation of a biological effect include those that signal the target cell through a tyrosine kinase, such as a src-family tyrosine kinase or a JAK family kinase, through a phosphatidylinositol 3-OH kinase.

Accordingly, target cell directed moieties that interact with a receptor on the surface of a target cell include, but are not limited to, natural or non-natural ligands of the receptor and antibodies that bind the receptor. Target cell directed moieties of the present invention include, but are not limited to, those that interact with CD3, CD28, CD2, MHC class I complex (including dimer, tetramer, multimer) loaded with peptide, MHC class Il complex (including dimer, tetramer, multimer) loaded with peptide, T cell receptor complexes (including alpha-beta and gamma-delta), CD 16, CD45, CD25, CD27, ICOS, CD40, CD40L, CTLA-4, OX-40, OX40L, CD30, CD30L, CD137, 4-1-BBL, B7.1, B7.2, Fas, FasL, TRAIL, DR4, DR5, DR3, TNFR1, TNFR2, chemokine receptors, receptors of cytokines (e.g., IL-1, IL-2, IL-3, IL-4, IL-5, IL-6, IL-7, IL-10, IL12P35, IL12P40, IL12P70, IL-13, IL-15, IL-18, IL-23, IL-27, TNF-alpha, TNF-beta, TGF-beta, IFN-gamma, GM-CSF), common gamma chain of IL-2 receptor and any associated components of cytokine receptors. Additional examples of target cell directed moieties for use in stimulation of a biological effect include those that interact with TNF-R, LT-βR, Her-2, CD19, IgM, CD20, CD21 and CD22. In some instances, target cell directed moieties of the invention include those that upon interaction with the target cell receptor result in target cell stimulation through a tyrosine kinase, such as a src-family tyrosine kinase or a JAK family kinase, through a phosphatidylinositol 3-OH kinase. In some instances, target cell directed moieties include a specific antigen, or fragment thereof, including tumor antigen and antigen associated with an infectious disease, such as a viral antigen.

Target cell directed moieties also include lectins, including lectins which can function as mitogens. In some embodiments, lectins which bind particular cell surface receptors, for example, through interaction with glycosylated moieties on the particular receptor, can be used to induce aggregation of the receptor. Thus, the lectin is a target cell directed moiety that can contribute to stimulation of a biological effect in the target cell. Generally, lectins are glycoproteins that can be extracted from plants, seeds and other sources, and many are commercially available. In some cases, the lectins are biotinylated. Examples of lectins include, but are not limited to, Aleuria aurantia lectin, Amaranthus caudatus lectin, Bauhinia purpurea lectin, Concanavalin A (Con A), Succinylated Con A, Datura stramonium lectin, Dolichos biflorus agglutinin, Erythrina cristagalli lectin, Euonymus europaeus lectin, Galanthus nivalis lectin, Griffonia (Bandeiraea) simplicifolia lectin I (GSL I, BSL I), GSL I-isolectin B₄, Griffonia (Bandeiraea) simplicifolia lectin II (GSL II, BSL II), Hippeastrum hybrid lectin, Jacalin, Lens culinaris agglutinin, Lotus tetragonolobus lectin, Lycopersicon esculentum lectin, Maackia amurensis lectin I (MAL I), Maackia amurensis lectin II (MAL II), Madura pomifera lectin, Narcissus pseudonarcissus lectin, Peanut agglutinin, Phaseolus vulgaris agglutinin (PHA-E+L), Phaseolus vulgaris erythroagglutinin (PHA-E), Phaseolus vulgaris leucoagglutinin (PHA-L), Pisum sativum agglutinin, Psophocarpus tetragonolobus lectin I (PTL I), Psophocarpus tetragonolobus lectin II (PTL II), Ricinus communis agglutinin I (RCA₁₂₀), Ricinus communis agglutinin II (ricin, RCA₆o), Sambucus nigra lectin, Solanum tuberosum lectin, Sophorajaponica agglutinin, Soybean agglutinin, Ulex europaeus agglutinin I (UEA I), Vicia villos lectin, Wheat germ agglutimn (WGA), Succinylated WGA, and Wisteria floribunda lectin.

As described herein, the target cell directed moiety can be included within or attached to another molecule as long as the target cell directed moiety portion of such a hybrid molecule is capable of interacting with the receptor on the target cell. Such molecules include target cell directed moiety—immunoglobulin (Ig) fusion proteins, for example, hybrid molecules containing a target cell directed moiety linked to an Fc fragment of an Ig. Ig fusion proteins are known in the art, including those that contain an Fc fragment that comprises the hinge, CH2 and CH3 regions of human IgG molecules. See, for example, U.S. Pat. No. 5, 116,964; Linsley et al. (1991) J Exp. Med. 173:721-730; Linsley et al. (1991) J Ex Med 174:561-569. Fusion proteins within the scope of the invention can be prepared by expression of a nucleic acid encoding the fusion protein in a variety of different systems known in the art and by other means known in the art.

Target cell directed moieties or molecules containing target cell directed moieties that are polypeptides will contain amino acid side chain moieties containing functional groups such as amino, carboxyl, or sulfhydryl groups that will serve as sites for coupling the target cell directed moiety to the linker. Residues that have such functional groups may be added to the target cell directed moiety if the target cell directed moiety does not already contain these groups. Such residues may be incorporated by solid phase synthesis techniques or recombinant techniques, both of which are well known in the peptide synthesis arts. In the case of target cell directed moieties or molecules containing target cell directed moieties that are carbohydrate or lipid, functional amino and sulfhydryl groups may be incorporated therein by conventional chemistry. For instance, primary amino groups may be incorporated by reaction with ethylenediamine in the presence of sodium cyanoborohydride and sulfhydryls may be introduced by reaction of cystamine dihydrochloride followed by reduction with a standard disulfide reducing agent. In a similar fashion, the linker molecule or the moiety on the RBC may also be derivatized to contain functional groups if it does not already possess appropriate functional groups.

In some embodiments, a ligand can be coupled to the surface of the complex which to direct the complex to a particular cell, organ, tissue and/or site within an individual. Such a ligand may serve to increase up-take of the complex by a particular organ or tissue. Directing ligands can be coupled to the complex surface through any of the means described herein for the target cell directed moiety. Complexes with coupled directing ligands may or may not have a target cell directed moiety also coupled. Example of such directing ligands include, but are not limited to, ligands which direct the complex to cells and/or tissue of the immune system, such as lymph nodes and spleen. complexes preferentially directed to cells and/tissue of the immune system include those containing antigen(s) to which an immune response is desired. Examples of ligands which direct the complex to the lymph nodes are CD62L and LFA-1. Complexes directed to the lymph nodes may further include an antigen linked to a Tat polypeptide of HIV to facilitate processing of the antigen.

In some embodiments, the disclosed complexes are loaded with an agent (e.g., a drug or antigen) and can serve as a delivery vehicle for the agent. As used herein, the term “loading” refers to introducing into or onto a disclosed complex at least one agent. In some embodiments, the agent is loaded by becoming internalized into the cell. In another embodiment, the agent is loaded by becoming coupled onto the surface of the cell and/or embedded in the membrane of the cell. Loading of a complex with more than one agent may be performed such that the agents are loaded individually (in sequence) or together (simultaneously or concurrently). Loading can occur before, during and/or after the target cell directed moiety is coupled to the surface of the complex. Loading is generally performed in a procedure separate from the procedure coupling a target cell directed moiety to the surface of the complex, however, in some cases, the procedures can be concurrent. Agents may be first admixed at the time of contact with the cells or prior to that time.

As used herein and in this context, an “agent” includes but is not limited to an atom or molecule, wherein a molecule may be inorganic or organic, a biological effector molecule and/or a nucleic acid encoding an agent such as a biological effector molecule, a protein, a polypeptide, a peptide, a nucleic acid, a peptide nucleic acid (PNA), a virus, a virus-like particle, a nucleotide, a ribonucleotide, a synthetic analogue of a nucleotide, a synthetic analogue of a ribonucleotide, a modified nucleotide, a modified ribonucleotide, an amino acid, an amino acid analogue, a modified amino acid, a modified amino acid analogue, a steroid, a proteoglycan, a lipid, a fatty acid and a carbohydrate.

As used herein, the term “biological effector molecule” or “biologically active molecule” refers to an agent that has activity in a biological system, including, but not limited to, a protein, polypeptide or peptide including, but not limited to, a structural protein, an enzyme, a cytokine (such as an interferon and/or an interleukin), a growth factor, an anti-apoptosis agent, an antigen, an antibiotic, a polyclonal or monoclonal antibody, or an effective part thereof, such as an Fv fragment, which antibody or part thereof may be natural, synthetic or humanized, a peptide hormone, a receptor, and a signaling molecule. As described herein, included within the term “immunoglobulin” are intact immunoglobulins as well as antibody fragments such as Fv, a single chain Fv (scFv), a Fab or a F (ab′)₂.

In some embodiments, the disclosed cell-targeting complexes are loaded with agents that promote Th1/Th2 cell growth, including, for example, IL-2, IL-7, IL-15, IL-18, IL-23, IL-27 and the like. In some embodiments, the RBCs are loaded with agents that promote Thl/Th2 cell differentiation, including, for example, IL-4, IL-12 and the like. In some embodiments, the disclosed cell-targeting complexes are loaded with anti-apoptosis agents including, for example, cellular FLICE (FADD-like IL-1 beta-converting enzyme) inhibitory protein (cFLIP), cIAP (inhibitor of apoptosis protein) 1 and 2.

In some embodiments, the compositions and/or methods of the invention involve disclosed cell-targeting complexes which are a) coupled to target cell directed moieties such as MHC I or MHC II tetramers loaded with a specific peptide or antigen specific for B cells and b) loaded with an agent such as an antigen or drug, e.g., FasL, TRAIL, TNF-alpha, IL-2, IL-15, IL-18, IL-23, or IL-27. Such compositions can thus direct the agent to the targeted B cell.

Loading may be performed by a procedure known in the art, such as a procedure selected from the group consisting of: iontophoresis, electroporation, sonoporation, microinjection, calcium precipitation, membrane intercalation, microparticle bombardment, lipid-mediated transfection, viral infection, osmosis, dialysis, including hypotonic dialysis, osmotic pulsing, osmotic shock, diffusion, endocytosis, phagocytosis, crosslinking to a red blood cell surface component, chemical crosslinking, mechanical perforation/restoration of the plasma membrane by shearing, single-cell injection, or a combination thereof. For example, a method and system for loading a cell with an agent is described in U.S. Pat. No. 6,495,351.

Sonoporation as a method for loading an agent into a cell is disclosed in, for example, Miller et al (1998) Ultrasonics 36:947-952. Iontophoresis uses an electrical current to activate and to modulate the diffusion of a charged molecule across a biological membrane, such as the skin, in a manner similar to passive diffusion under a concentration gradient, but at a facilitated rate. In general, iontophoresis technology uses an electrical potential or current across a semipermeable barrier. By way of example, iontophoresis technology and references relating thereto is disclosed in WO 97/49450. Another method for loading agents in RBCs is electroporation. Electroporation has been used for encapsulation of foreign molecules in different cell types including red blood cells as described in Mouneimne et al. (1990) FEBS 275, No. 1, 2, pp. 117-120 and in U.S. Pat. No. 5,612,207. The process of electroporation involves the formation of pores in the cell membranes by the application of electric field pulses across a liquid cell suspension containing the cells. During the poration process, cells are suspended in a liquid media and then subjected to an electric field pulse. The medium may be electrolyte, non-electrolyte, or a mixture of electrolytes and non-electrolytes. The strength of the electric field applied to the suspension and the length of the pulse (the time that the electric field is applied to a cell suspension) varies according to the cell type, as is known in the art.

Loading may also take place by way of hypotonic dialysis. The dialysis devices used may be conventional dialysis devices as known in the art. Dialysis devices work on the principle of osmotic shock, whereby loading of an agent into red blood cell, is facilitated by the induction of sequential hypotonicity and recovery of isotonicity. The term “osmotic shock” is intended herein to be synonymous with the term “hypotonic dialysis” or “hypoosmotic dialysis.” An exemplary osmotic shock/hypotonic dialysis method is described in Eichler et al. (1986) Res. Exp. Med. 186:407-412. For example, washed red blood cells are suspended in 1 ml of PBS (150 mMNaCl, 5 mM K₂HPO₄/KH₂PO₄, pH 7.4) to obtain a hematocrit of approximately 60%. The suspension is placed in dialysis tubing (molecular weight cut-off 12,000-14,000; Spectra-Por) and cells are dialyzed against 100 ml of 5 mM K₂HP0/KH₂PO, pH 7.4 for 90 minutes at 4° C., thereby swelling the cells and rendering them permeable to agents to be loaded. Resealing is achieved by further dialysis, e.g., for 15 minutes at 37° C. against 100 ml of PBS containing 10 mM glucose. Cells are then washed in ice cold PBS containing 10 mM glucose using centrifugation.

Alternatively, other osmotic shock procedures can be implemented such as described, for example, in U.S. Pat. No. 4,478,824. For example, a packed RBC or platelet fraction is incubated in a solution containing a compound (such as dimethyl sulphoxide (DMSO) or glycerol) which readily diffuses into and out of cells. The compound rapidly creates a transmembrane osmotic gradient by diluting the suspension of RBCs in the solution with a near-isotonic aqueous medium. By including an anionic agent in the medium which may be an allosteric effector of hemoglobin, such as inosine monophosphate or a phosphorylated inositol (e.g., inositol hexaphosphate), water diffuses into the cells, swelling the cells and increasing the permeability of the outer membranes of the cells. Thus, the method may be used to load cells with anionic agents, as the increase in the permeability is maintained for a period of time sufficient only to permit transport of the anionic agent into the cells and diffusion of readily-diffusing compounds out of the cells. However, this is generally not the method of choice where the desired agent to be loaded into cells is not anionic, or is anionic or polyanionic, but is not present in the near-isotonic aqueous medium in sufficient concentration to cause the needed increase in cell permeability without cell destruction. Other methods of loading RBCs or platelets with selected agents using an osmotic shock technique are described in U.S. Pat. No. 4,931,276.

It will be appreciated by one skilled in the art that combinations of methods may be used to facilitate the loading of an disclosed complex with agents of interest. Likewise, it will be appreciated that, when more than one agent is to be loaded, such as a first and second agent, the first and second agent may be loaded concurrently or sequentially, in either order.

The disclosed cell-targeting complexes may be concentrated to facilitate loading, coupling of the target cell directed moiety and/or administration of the RBCs or platelets. Methods for concentrating RBCs or platelets and other cells are known in the art. Similar methods may also be used in the separation and/or purification of the disclosed cell-targeting complexes before or after loading and/or coupling of target cell directed moiety to the surface. Such methods include, for example, filtration and/or centrifugation techniques. Concentration and/or purification methods can be used to prepare the disclosed cell-targeting complexes so that they are at a concentration or cell density useful for the desired purpose.

Since blood components have magnetization properties, magnetism has been used to separate and/or isolate blood components. Thus, other methods for separating and/or concentrating cells uses magnetic techniques. Magnetic separation is described in detail in U.S. Pat. Nos. 4,910, 148, 5,514,340, 5,567,326, 5,541,072, 4,988,618, 4,935,147, 6,132,607, 6, 129,848 and 6,036,857. In such methods, for example, magnetic beads or microbeads are coated with a molecule(s) suitable for specifically binding to an RBC or platelet, for example an antibody or other binding moiety capable of specifically binding to an RBC antigen, such as a molecule present on the surface of an RBC. RBCs or platelets are then mixed with such magnetic beads or microbeads and then transferred to a chamber where a magnetic field is applied to separate beads to which red blood cells are bound from other components. Alternatively, the beads may be provided within a collection device, and the collection device may be transformed into a separation device through the application of a magnetic field.

### Antibody Compositions

As described herein, the disclosed complexes may include an antibody that binds a receptor on the target cell, or the receptor binding portion of the antibody, as a target cell directed moiety. In such complexes, the anti-target cell antibody is coupled, either directed or indirectly, to the surface of disclosed cell-targeting complexes as described herein. As an example, the antibody can be labeled with biotin and coupled to the surface of a disclosed cell-targeting complexes through a biotin-avidin coupling.

Antibodies are understood to include various kinds of antibodies, including, but not necessarily limited to, naturally occurring antibodies, monoclonal antibodies, polyclonal antibodies, antibody fragments that retain antigen binding specificity (e.g., Fab, and F(ab′)₂) and recombinantly produced binding partners, single domain antibodies, hybrid antibodies, chimeric antibodies, single-chain antibodies, human antibodies, humanized antibodies, and the like. Generally, antibodies are understood to be reactive against a selected antigen on the surface of a cell if they bind with an affinity (association constant) of greater than or equal to 10⁻⁶ M.

Polyclonal antibodies against selected antigens on the surface of cells may be readily generated by one of ordinary skill in the art from a variety of warm-blooded animals such as-horses, cows, various fowl, rabbits, mice, or rats. In some cases, human polyclonal antibodies against selected antigens may be purified from human sources.

Monoclonal antibodies specific for selected antigens on the surface of cells may be readily generated using conventional techniques (see, for example, Harlow et al., 1988, supra, and U.S. Pat. Nos. RE 32,011, 4,902,614, 4,543,439, and 4,411,993). Hybridoma cells can be screened immunochemically for production of antibodies specifically reactive with an antigen, and monoclonal antibodies can be isolated. Other techniques may also be utilized to construct monoclonal antibodies (see, for example, Huse et al. (1989) Science 246:1275-1281; Sastry et al. (1989) Proc. Natl. Acad. Sci. USA 86:5728-5732; Alting-Mees et al. (1990) Strategies in Molecular Biology 3:1-9).

Similarly, binding partners may be constructed utilizing recombinant DNA techniques. For example, the genes which encode the variable region from a hybridoma producing a monoclonal antibody of interest are amplified using nucleotide primers for the variable region. These primers may be synthesized by one of ordinary skill in the art, or may be purchased from commercially available sources. The primers may be utilized to amplify-heavy or light chain variable regions, which may then be inserted into appropriate expression vectors. These vectors may then be introduced into cells, for example E. coli cells, for expression. Utilizing these techniques, large amounts of a single-chain protein containing a fusion of the H and V domains may be produced (see, for example, Bird et al. (1988) Science 242:423-426). In addition, such techniques may be utilized to change a “murine” antibody to a “human” antibody, without altering the binding specificity of the antibody.

As used herein, a “single domain antibody” (dAb) is an antibody which is comprised of a V_{H} domain, which reacts immunologically with a designated antigen. A dAb does not contain a domain, but may contain other antigen binding domains known to exist in antibodies, for example, the kappa and lambda domains. Methods for preparing dAbs are known in the art. See, for example, Ward et al. (1989) Nature 341:544-546. Antibodies may also be comprised of VH and V_{L} domains, as well as other known antigen binding domains. Examples of these types of antibodies and methods for their preparation are known in the art (see, e.g., U.S. Pat. No. 4,816,467).

Further exemplary antibodies include “univalent antibodies”, which are aggregates comprised of a heavy chain/light chain dimer bound to the Fc (i.e., constant) region of a second heavy chain. This type of antibody generally escapes antigenic modulation. See, e.g., Glennie et al. (1982) Nature 295:712-714.

Antibodies can be fragmented using conventional techniques and the fragments (including “Fab” fragments) screened for utility in the same manner as described above for whole antibodies. The “Fab” region refers to those portions of the heavy and light chains which are roughly equivalent, or analogous, to the sequences which comprise the branch portion of the heavy and light chains, and which have been shown to exhibit immunological binding to a specified antigen, but which lack the effector Fc portion. “Fab” includes aggregates of one heavy and one light chain (commonly known as Fab′), as well as tetramers containing the 2H and 2L chains (referred to as F(ab)₂), which are capable of selectively reacting with a designated antigen or antigen family. Methods of producing Fab fragments of antibodies are known within the art and include, for example, proteolysis, and synthesis by recombinant techniques. For example, F(ab′)₂ fragments can be generated by treating antibody with pepsin. The resulting F(ab′)₂ fragment can be treated to reduce disulfide bridges to produce Fab′ fragments. “Fab” antibodies may be divided into subsets analogous to those described herein, i.e., “hybrid Fab”, “chimeric Fab”, and “altered Fab”.

“Hybrid antibodies” are antibodies wherein one pair of heavy and light chains is homologous to those in a first antibody, while the other pair of heavy and light chains is homologous to those in a different second antibody. Typically, each of these two pairs will bind different epitopes, particularly on different antigens. This results in the property of “divalence”, i.e., the ability to bind two antigens simultaneously. Such hybrids may also be formed using chimeric chains, as set forth herein.

“Altered antibodies” are antibodies in which the naturally occurring amino acid sequence in a vertebrate antibody has been varied. Utilizing recombinant DNA techniques, antibodies can be redesigned to obtain desired characteristics. The possible variations are many, and range from the changing of one or more amino acids to the complete redesign of a region, for example, the constant region. Changes in the constant region, in general, to attain desired cellular process characteristics. Changes in the variable region may be made to alter antigen binding characteristics. The antibody may also be engineered to aid the specific delivery of a molecule or substance to a specific cell or tissue site. The desired alterations may be made by known techniques in molecular biology, e.g., recombinant techniques, site directed mutagenesis, and other techniques.

By “humanized” is meant alteration of the amino acid sequence of an antibody so that fewer antibodies and/or immune responses are elicited against the humanized antibody when it is administered to a human. For the use of the antibody in a mammal other than a human, an antibody may be converted to that species format.

“Chimeric antibodies”, are antibodies in which the heavy and/or light chains are fusion proteins. Typically the constant domain of the chains is from one particular species and/or class, and the variable domains are from a different species and/or class. The invention includes chimeric antibody derivatives, i.e., antibody molecules that combine a non-human animal variable region and a human constant region. Chimeric antibody molecules can include, for example, the antigen binding domain from an antibody of a mouse, rat, or other species, with human constant regions. A variety of approaches for making chimeric antibodies have been described and can be used to make chimeric antibodies containing the immunoglobulin variable region which recognizes selected antigens on the surface of differentiated cells or tumor cells. See, for example, Morrison et al. (1985) Proc. Natl. Acad. Sci. U.S.A. 81:6851; Takeda et α/. (1985) Natωre 314:452; U.S. Pat. Nos. 4,816,567 and 4,816,397; European Patent Publications EP171496 and EP173494; United Kingdom patent GB 2177096B. sss

Bispecific/Multispecific antibodies may contain a variable region of an anti-target cell receptor antibody and a variable region specific for at least one antigen on the surface of an RBC or platelet. In other cases, Bispecific/Multispecific antibodies may contain a variable region of an anti-target cell receptor antibody and a variable region specific for a linker molecule. In other cases, Bispecific/Multispecific antibodies may contain a variable region specific for at least one antigen on the surface of an RBC or platelet and a variable region specific for a linker molecule. Bispecific/Multispecific antibodies may be obtained forming hybrid hybridomas, for example by somatic hybridization. Hybrid hybridomas may be prepared using the procedures known in the art such as those disclosed in Staerz et al. (1986, Proc. Natl. Acad. Sci. U.S.A. 83:1453) and Staerz et al. (1986, Immunology Today 7:241). Somatic hybridization includes fusion of two established hybridomas generating a quadroma (Milstein et al. (1983) Nature 305:537-540) or fusion of one established hybridoma with lymphocytes derived from a mouse immunized with a second antigen generating a trioma (Nolan et al. (1990) Biochem. Biophys. Ada 1040:1-11). Hybrid hybridomas are selected by making each hybridoma cell line resistant to a specific drug-resistant marker (De Lau et al. (1989) J. Immunol. Methods 117:1-8), or by labeling each hybridoma with a different fluorochrome and sorting out the heterofluorescent cells (Karawajew etα/. (1987) J Immunol. Methods 96:265-270).

Bispecific/Multispecific antibodies may also be constructed by chemical means using procedures such as those described by Staerz et al. (1985) Nature 314:628 and Perez et al. (1985) Nature 316:354. Chemical conjugation may be based, for example, on the use of homo-and heterobifunctional reagents with E-amino groups or hinge region thiol groups. Homobifunctional reagents such as 5,5′-dithiobis (2-nitrobenzoic acid) (DNTB) generate disulfide bonds between, the two Fabs, and O-phenylenedimaleimide (O-PDM) generate thioether bonds between the two Fabs (Brenner et al. (1985) Cell 40:183-190, Glennie et al. (1987) J Immunol. 139:2367-2375). Heterobifunctional reagents such as N-succinimidyl-3-(2-pyridylditio) propionate (SPDP) combine exposed amino groups of antibodies and Fab fragments, regardless of class or isotype (Van Dijk et al. (1989) Int. J. Cancer 44:738-743).

Bifunctional antibodies may also be prepared by genetic engineering techniques. Genetic engineering involves the use of recombinant DNA based technology to ligate sequences of DNA encoding specific fragments of antibodies into plasmids, and expressing the recombinant protein. Bispecific/Multispecific antibodies can also be made as a single covalent structure by combining two single chains Fv (scFv) fragments using linkers (Winter et al. (1991) Nature 349:293-299); as leucine, zippers coexpressing sequences derived from the transcription factors fos and jun (Kostelny et al. (1992) J. Immunol. 148:1547-1553); as helix-turn-helix coexpressing an interaction domain of p53 (Rheinnecker et al. (1996) J. Immunol. 157:2989-2997), or as diabodies (Holliger et al. (1993) Proc. Natl. Acad. Sci. U.S.A. 90-: 6444-6448).

A tetrameric immunological complex may be prepared by mixing a first monoclonal antibody which is capable of binding to at least one receptor on the surface of a target cell and a second monoclonal antibody which is capable of binding to a moiety on an RBC or platelet. The first and second monoclonal antibodies are from a first animal species. The first and second antibodies are reacted with monoclonal antibodies of a second animal species which are directed against the Fc-fragments of the antibodies of the first animal species. The first and second antibody may also be reacted with the F(ab′)₂ fragments of monoclonal antibodies of a second animal species which are directed against the Fc-fragments of the antibodies of the first animal species. See, for example, U.S. Pat. No. 4,868,109. For example, the first and second antibody may be reacted with an about equimolar amounts of the monoclonal antibodies of the second animal species or of the F(ab′)₂ fragments thereof.

Antibodies may be selected for use in the antibody compositions based on their ability to stimulate the desired biological effect in the target cell. In some embodiments, anti-target cell antibodies include antibodies specific for the antigens CD3 and CD28 which are present on the surface of human CD4+ T cells. Monoclonal antibodies against CD3, and CD28, in the antibody composition of the invention are used to stimulate a biological effect in T cells. Examples of monoclonal antibodies specific for CD3 and CD28. are OKT3 and L293, respectively, and additional examples are in the art.

### Formulations and Routes of Administration

In some embodiments, the disclosed complexes are used in the preparation of medicaments, for treating the conditions described herein. These complexes are administered as pharmaceutically acceptable compositions. The complexes may be administered by any suitable means, including, but not limited to, intravenously, parenterally or locally. The complexes can be administered in a single dose by bolus injection or continuous infusion or in several doses over selected time intervals in order to titrate the dose.

The particular administration mode selected will depend upon the particular composition, treatment, cells involved, etc. The volume will depend upon, for example, the type of cell administered, the disorder treated and the route of administration.

As used herein, “pharmaceutically acceptable excipient” includes any material which, when combined with an active ingredient of a composition, allows the ingredient to retain biological activity and without causing disruptive reactions with the subject's immune system. Various pharmaceutically acceptable excipients are well known in the art.

Exemplary pharmaceutically acceptable excipients include sterile aqueous or non-aqueous solutions and suspensions. Examples include, but are not limited to, any of the standard pharmaceutical excipients such as a phosphate buffered saline solution, water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Compositions comprising such excipients are formulated by well known conventional methods (see: for example, Remington's Pharmaceutical Sciences, 18th Ed., Mack Publishing Co.).

### Specific Embodiments

Embodiment 1. A cell-targeting complex, comprising a non-nucleated or nucleated cell comprising cell surface proteins that have been thiolated and coated with a streptavidin, avidin, or a derivative or analogue thereof with high affinity for biotin, using a sulfo-SMCC linker, and then coated with one or more biotinylated antibodies.

Embodiment 2. The cell-targeting complex of embodiment 1, wherein the cell is a red blood cell (RBC) or platelet.

Embodiment 3. A cell-targeting complex, comprising a multilayer alginate hydrogel beads comprising a cell surface that has been coated with a polyanionic protein using a polycation selected from the group consisting of as poly-D-lysine (PDL), poly-L-lysine (PLL), poly-L-ornithine (PLO), and any combination thereof, wherein the coated polyanionic protein is thiolated and streptavidinylated with a sulfo-SMCC linker, and then coated with one or more biotinylated antibodies.

Embodiment 4. The cell-targeting complex of embodiment 3, wherein the polyanionic protein is selected from the group consisting of collagen, gelatin, laminin, fibronectin, or any combination thereof.

Embodiment 5. The cell-targeting complex of any one of embodiments 1 to 4, wherein the cell surface proteins or coated polyanionic proteins have been thiolated with Traut's reagent (2-Iminothiolane).

Embodiment 6. The cell-targeting complex of any one of embodiments 1 to 5, wherein the one or more biotinylated antibodies comprise anti-CD3 and anti-CD28 antibodies.

Embodiment 7. The cell-targeting complex of embodiment 6, wherein the cell-targeting complex is loaded with cytokines and/or growth factors configured to enhance activation and proliferation of T cells.

Embodiment 8. The cell-targeting complex of any one of embodiments 1 to 7, wherein the one or more biotinylated antibodies bind tumor antigens.

Embodiment 9. The cell-targeting complex of embodiment 8, wherein the cell-targeting complex is loaded with costimulatory molecules, death receptors, chemokines, and/or cytokines configured to kill cancer cells.

Embodiment 10. A method for activating an expanding T cells in vitro, comprising contacting the T cells with the cell-targeting complex of embodiment 6 or 7.

Embodiment 11. A method for treating cancer in a subject, comprising administering to the subject an effective amount of the cell-targeting complex of embodiment 8 or 9.

Embodiment 12. A method for transporting an agent in a system to a cell, the method comprising loading the cell-targeting complex of any one of embodiments 1 to 9 with the agent, and contacting the system with the loaded cell-targeting complex.

Embodiment 13. The method of embodiment 12, wherein the agent is oxygen, a free radical enzyme, a nutrient, or any combination thereof.

Embodiment 14. The method of embodiment 13, wherein the cell-targeting complex is pre-loaded with an artificial oxygen carrier and then exposed to oxygen prior to or after contact with the system.

Embodiment 15. The method of embodiment 14, wherein the artificial oxygen carrier comprises perfluorocarbon, HEMOGLOBIN-BASED OXYGEN CARRIERS, X80, FC75, FC43, FC17 (perfluorotetrahydrofuran, perfluorotributylamine), perfluorodecaline, or perfluoromethyl decaline.

Embodiment 16. The method of any one of embodiments 12 to 15, wherein the agent is a viral vector.

Embodiment 17. The method of any one of embodiments 12 to 16, wherein the system is a human or animal subject.

Embodiment 18. A method for expanding, differentiating, or killing a cell in a culture system, comprising culturing the cell in the presence of the cell-targeting complex of any one of embodiments 1 to 9.

Embodiment 19. The method of embodiment 18, wherein the cell-targeting complex is configured as a feeder layer for the cell.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Example Protocol for Producing RBCs Coated With Antibodies

In this example, modified RBC are prepared by the attachment of Streptavidin molecules on the surface of RBCs followed by the addition of biotinylated anti-CD3 and anti-CD28 antibodies. Streptavidinylation process involves the chemical modification or thiolation of surface proteins of RBCs using a chemical reagent which will convert surface amines to thiols. This chemical reagent is a cyclic thioimidate compound for sulfhydryl addition. In the present study Traut's reagent is used for sulfhydryl addition on RBC. It reacts spontaneously and efficiently with primary amines at pH 7-9, introducing sulfhydryl groups while maintaining charge properties similar to the original amino group. The resulting surface thiols now can be linked to Streptavidin molecules using a linker, sulfo-SMCC, a heterobifunctional crosslinker that contains N-hydroxysuccinimide (NHS) ester and maleimide groups that allow covalent conjugation of amines in streptavidin molecules and sulfhydryls in thiolated RBCs. Once streptavidin is attached on to the RBC surface, biotinylated antibodies are added to prepare the RBC-SA-3/28 complexes which will be used for T cell activation. The following is an example protocol:

### Step 1: Preparation of Streptavidinylated-RBC

Coupling of streptavidin to RBC is composed of three steps. Step A, preparation of maleimide-streptavidine complexes (SA-SMCC); Step B, Red Blood Cell (RBC) reduction by Traut's reagent; and Step C, Loading of maleimide SA onto Reduced-RBCs (SA-RBC). Steps A and B are processed simultaneously.

Step-A: Preparation of Maleimide-SA Complexes (SA-SMCC): Streptavidin is dissolved at 1-10 mg/mL in Hank's Balance Salt Solution (HBSS) and Sulfo-SMCC (S-SMCC) is dissolved separately at 1-10 mg/ml in pre-warmed DPBS (37° C.). Sulfo-SMCC [100 μL at 1-10 mg/mL] is added to each 1-10 mg/ml of streptavidin solution and mixed for 2 hours at room temperature. Un-reacted S-SMCC and free sulfo-N-hydroxsuccinamide is removed by filtration under sterile conditions using YM-10 Micron centrifugal filter devices as per manufacturers guidelines. Following the washing steps the concentration of the SA-SMCC was adjusted to 2-20 mg/ml using sterile HBSS.

Step-B: Red Blood Cell (RBC) Reduction by Traut's reagnet: RBC are isolated from peripheral blood and then washed twice with cold HBSS. Discard as much supernatant as possible and disturb the pellet thoroughly. Add 2 ml 1-10 mM sterile filtered Traut's reagent solution per 1-10×10⁹ RBC, incubate it at RT for 1-10hrs in dark with continuous mixing. At the end of incubation, wash the RBC twice with HBSS to remove excess Traut's reagent. Discard the supernatant and re-suspend the RBC in HBSS at a density of 2×10⁹/ml.

Step-C: Loading of maleimide SA onto Reduced-RBCs (SA-RBC): In a sterile polypropylene tube, mix 1-10 mg of SA-SMCC complex (prepared in step-1) per 1-10×10⁹ reduced RBC (prepared in step-2), mix well, protect from excessive sunlight with (aluminum foil or paper) and place on an end-to-end rotator at room temperature for 1 hour. At the end of 1 hour, wash the streptavidin coupled RBC thrice with HBSS to remove excess SA-SMCC. Discard the supernatant and re-suspend the RBC in HBSS at a density of 2×10⁹/ml.

### Confirm RBC Streptavidinylation by Flow Cytometry

Transfer 1-25×10⁶ RBC-Streptavidin conjugates (RBC-SA) conjugates and thiolated RBCs (as described in the table below) in to 15 mL tubes containing 3 mL of FACS buffer. Centrifuge at 1000 g for 5 minutes with brakes and discard the supernatant by decanting. Disrupt the pellet and add 50 μL of FACs buffer. Add FITC anti SA antibody as described in the table 2 into appropriate tubes and mix well. Incubate at 4° C. for 20 minutes. Add 4 mL of FACS buffer and centrifuge at 1000 g for 5 minutes with brakes. Discard the supernatant and resuspend in 1.5 mL of FACS buffer and transfer 250 μL per well in to a 96 well plate. Acquire on the Flow Cytometer.

TABLE 1
FITC anti SA antibody labelling conditions

| Tube # | RBCs | FITC anti SA antibody |
|---|---|---|
| 1 | Reduced / Sulfhydryl RBCs | None |
| 2 | Reduced / Sulfhydryl RBCs | 2 μL |
| 3 | RBC-SA conjugates | None |
| 4 | RBC-SA conjugates | 2 μL |

### Step 2: Preparation of Human Anti-CD3 and Anti-CD28 Conjugated RBC

Ratio; 50×10⁶ SA-RBC: 12.5 μg anti-CD3: 12.5 μg anti-CD28 in 100 μl HBSS SA-RBC is at 2 B/mL in HBSS+/+ Therefore, add 25 uL to a 1.7 mL tube (50 M) Biotinylated anti-CD3/CD-28 at 0.5 mg/mL, 100 ug. In a sterile 1.7 ml polypropylene tube, prepare the antibody cocktail by adding HBSS, biotinylated anti CD3 (25 uL), biotinylated anti CD28 (25 uL) in the order as stated. Mix by gentle vortex. Prepare RBC-3/28 by adding the antibody cocktail to SA-RBCs (25 uL) in a 1.7 mL tube. Mix quickly by inverting 3-4 times. Mix on the Roto mix (at setting of 4.5) with tape and initials at RT for 30 minutes. Following incubation, centrifuge RBC-3/28 suspension at 350 g/RT for 10 minutes without brakes. Wash RBC-3/28 twice with HBSS: fill the tube and centrifuge at 350 g/RT for 10 minutes without brakes. Do not decant; gently remove supernatant with a pipette and resuspend in 25 uL volume of HBSS for counting. Adjust the SA-RBC cell density to 2B/mL (if 50 M cells are there, in 50 uL volume)

### Results

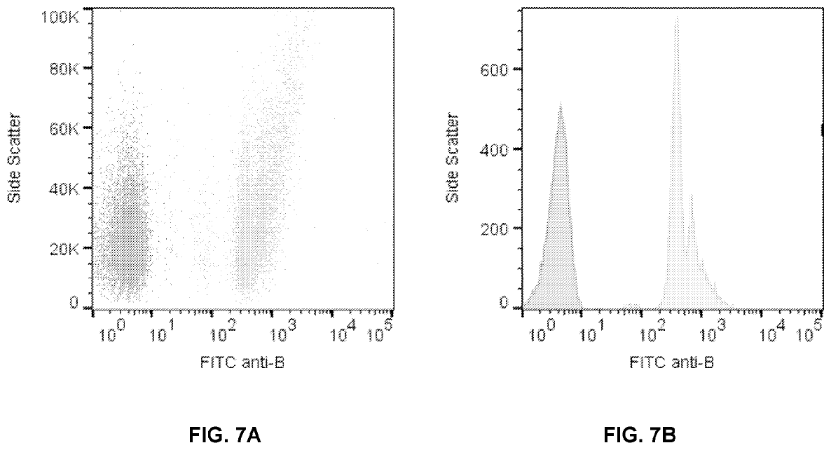

FIGS. 7A and 7B show assessment of Streptavidinylated-RBC (SA-RBC) conjugation. Shown is flow cytometry analysis of FITC stained RBCs conjugated with Streptavidin. Shown are unlabeled RBCs (mean fluorescence intensity (MFI); 4.28) and FITC labelled SA-RBC conjugates (MFI; 648). FIG. 7A shows a dot plot representation FIG. 7B is a histogram representation of data. As shown in the graphs FITC RBC-SA conjugates has about >100-fold MFI compared to the unstained control.

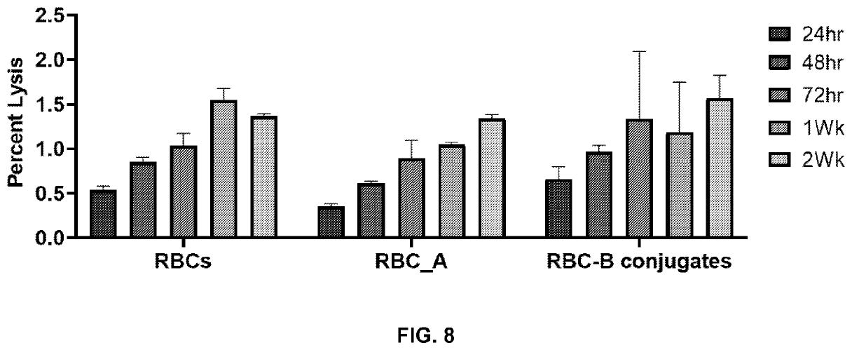

FIG. 8 shows stability assessment of RBCs, Thiolated-RBC (RBC_A) and RBC-SA (RBC-B conjugates). Shown is the percent Lysis of RBC, RBC_A and RBC-B conjugates stored in RB RBC storage buffer at 2×10⁹ cells/mL cell density up to 2 weeks at 4° C. As depicted in the figure <2.5% lysis was observed when RBCs are stored at the conditions mentioned above up to 2 weeks' time.

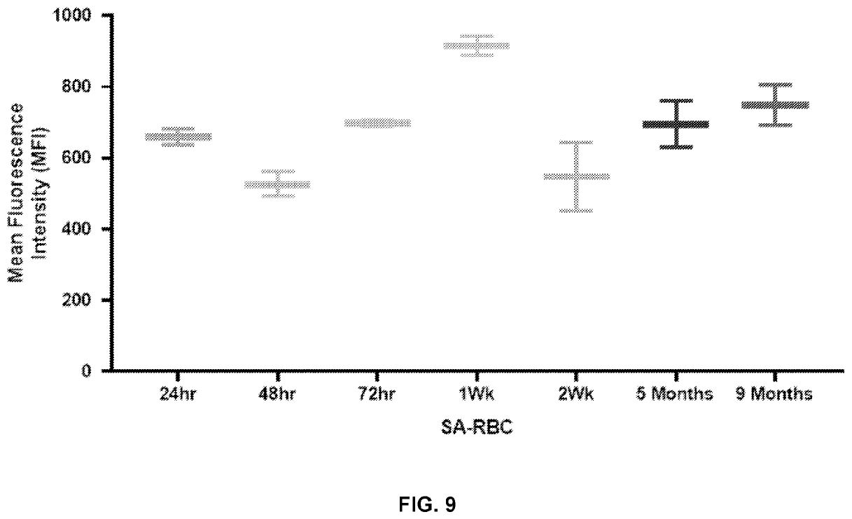

FIG. 9 shows stability assessment of RBC-SA (RBC-B) conjugates. Shown in s the mean fluorescence intensity of RBC-SA conjugates stored in RB RBC storage buffer at 2×109 cells/mL cell density up to 9 months. Statistical analysis was performed using ordinary one-way ANOVA followed by Tukey's post hoc test for multiple comparisons. P<0.05 was considered as significant. There was no statistically significant difference between samples at each time point. As depicted in the figure, when RBC-SA conjugates are stored at conditions mentioned above, they are stable up to 2 weeks.

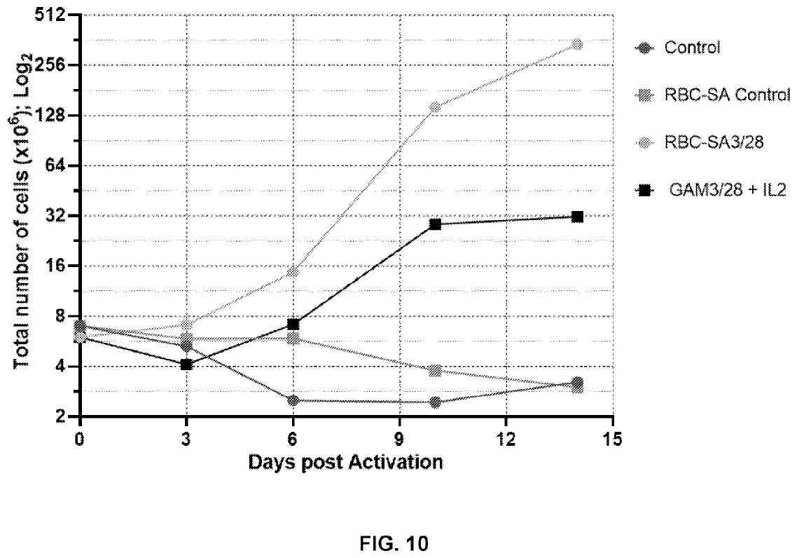

FIG. 10 shows activation/proliferation of macrophages depleted human PBMC using hu-anti-CD3 and hu-anti-CD28 conjugated to GAM beads (Dynabeads) vs RBC. Cell growth is shown as Log 2 cell number (×106) over the 14 days of post activation.

Macrophage depleted human PBMCs were activated by Streptavidinylated RBCs, conjugated with biotinylated anti-CD3 and biotinylated anti-CD28 antibodies (RBC-SA3/28) at day 0. As a conventional activation control, macrophage depleted PBMCs were activated by Goat anti mouse magnetic beads coated with anti-CD3 and anti-CD28 antibodies (GAM3/28) in presence of IL-2 (30 U/mL). Untreated and cells treated with Streptavidinylated RBCs were used as negative assay controls. At day 3 & 6, number of cells were counted and cultures were re-stimulated with RBC-SA3/28 and GAM3/28+IL-2 (30 U/mL). Negative controls were treated with RBC-SA (RBC-SA control only). On day 10, cells were counted and further re-stimulation was done by treating the RBC-SA3/28 activated cells with Streptavidin-Biotin conjugated anti-CD3 and anti-CD28 (SA3/28) complexes. Positive control was re-stimulated by GAM3/28 along with IL-2 (10 U/mL) while negative control was treated with RBC-SA. After day 6, each day culture volume was doubled using complete media.

Orange solid circles represent the cells in culture stimulated with RBC-SA3/28 at day 0. Black solid squares represent the cells in culture stimulated with GAM3/28 along with IL-2. Red solid squares represent the cells in culture stimulated with RBC-SA only while blue solid circles represents unstimulated cells in culture.

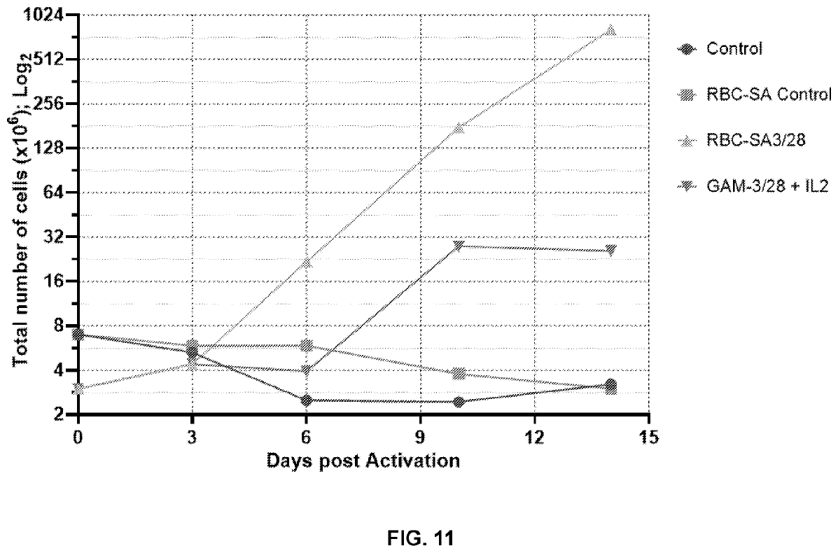

FIG. 11 shows activation/proliferation of human Pan-T cells using hu-anti-CD3 and hu-anti-CD28 conjugated to GAM beads (Dynabeads) vs RBC. Cell growth is shown as Log 2 cell number (×10⁶) over the 14 days of post activation.

Human Pan T cells were isolated and activated by Streptavidinylated RBCs, conjugated with biotinylated anti-CD3 and biotinylated anti-CD28 antibodies (RBC-SA3/28) at day 0. As a conventional positive control, Pan T cells were activated by Goat anti mouse magnetic beads coated with biotinylated anti-CD3 and biotinylated anti-CD28 antibodies (GAM3/28) along with IL-2 (30 U/mL). Untreated cells and cells treated with Streptavidinylated-RBCs were used as negative controls. At day 3 & 6, number of cells were counted and cultures were re-stimulated with RBC-SA3/28 and GAM3/28+IL-2 (30 U/mL). Negative controls were treated with RBC-SA (RBC-SA control only). On day 10, cells were counted and further re-stimulation was done by treating the RBC-SA3/28 activated cells with Streptavidin-Biotin conjugated anti-CD3 and anti-CD28 (SA3/28) complexes. Positive control was re-stimulated by GAM3/28 along with IL-2 (10 U/mL) while negative control was treated with RBC-SA. After day 6, each day culture volume was doubled using complete media.

Green solid triangles represent the cells in culture stimulated with RBC-SA3/28 at day 0. Purple solid triangles represent the cells in culture stimulated with GAM3/28 along with IL-2. Red solid squares represent the cells in culture stimulated with RBC-SA only while blue solid circles represents unstimulated cells in culture.

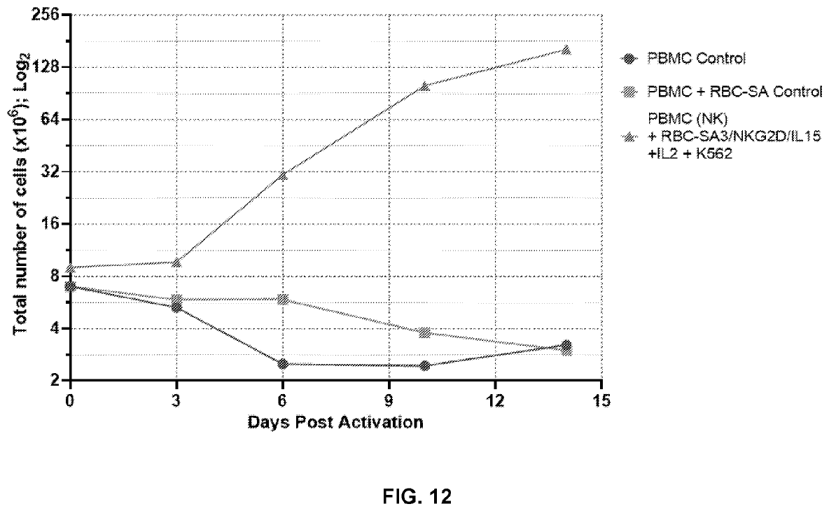

FIG. 12 shows activation/proliferation of NK cells in PBMC cultures using streptavidinylated RBCs coated with hu-anti-CD3, hu-anti-NKG2D and IL-15 plus soluble ILD2 and irradiated K562 cells. Plot depicting human PBMC (NK) growth as Log 2 cell number (×10⁶) versus days post activation up to 14 days

In order to activate and expand NK cells, human PBMCs were isolated and activated, activation was done by Streptavidinylated RBCs (1:5, PBMC: RBC ratio) conjugated with biotinylated anti-CD3 and biotinylated anti-NKG2D, biotinylated IL-15 (RBC-SA3/NKG2D/IL-15) along with IL-2 (30 U/mL) and irradiated K562 feeder cells (6:1, PBMCs: K562 ratio) at day 0. Untreated PBMCs and PBMC treated with Streptavidinylated RBCs were used as negative controls. At day 3, number of cells were counted and cultures were re-stimulated with RBC-SA3/NKG2D/IL-15 along with IL-2 at 10 U/mL. Negative control was treated with RBC-SA (PBMC+RBC-SA control only). At day 5, NK cells were re-stimulated with IL-2 at 10 U/mL and at day 6 cells were counted. At day 7, further re-stimulation was done by treating cells with IL-2 at 100 U/mL and biotinylated IL-15 at 5 ng/ml. Final re-stimulation of NK cells was performed at day 10 with the addition of IL-2 at 200 U/mL and biotinylated IL-15 at 10 ng/ml. Upon expanding the cells up to day 14, final cell count was performed and cultures were terminated.

Brown solid triangles represent the cells in culture stimulated with RBC-SA3/NKG2D/IL-15 along with IL-2 at day 0. Red solid squares represent the cells in culture stimulated with RBC-SA only while blue solid circles represents unstimulated cells in culture.

### Example 2: Example Protocol for Producing Multilayer Alginate Hydrogel Beads Coated With Antibodies

In this example, multilayer alginate hydrogel beads are prepared for streptavidinylation and coating with biotinylated antibodies.

Ultrapure Sodium Alginate powder is dissolved in 300 mOsmol NaCl solution at a concentration of 1.6% w/v and then filtered through a syringe filter. Ionically crosslinked calcium alginate microcapsules are fabricated by extruding this the solution of sodium alginate through a 400 μm diameter nozzle of the Buchi B-395 Pro Encapsulator or Nisco Modulator Micro-Encapsulator. The biological agents such as cytokines, and/or growth factor and/or, oxygenation agents and/or cytotoxic agent, are mixed into the sodium alginate solution at desired concentration prior to the encapsulation process in order to form the final therapeutic product. The microcapsules/microbeads are formed by using air dispersion principle where a controlled flow of filtered Ultrapure Nitrogen disperses a jet of alginate solution, along with its components, into droplets of defined size and shape which fall into the 300 mOsmol CaCl₂ gelling bath. The size of the microcapsules is precisely controlled by using optimized injection flow rates and pressure of the dispersion gas. The final product is collected in a sterile container containing 100 mM CaCl₂ and filtered through a 300 μm pore size filter. This is followed by a rinse with cell culture media to remove any trace of the CaCl₂ gelling solution. The microcapsules are then resuspended into culture medium and plated in gas permeable vessels. Next to coat these beads with coating agents viz., PDL and/or, PLL and/or, PLO and/or, Collagen and/or, Gelatin and/or, Laminin and/or, Fibronectin and/or, PDL+Laminin and/or, PDL+Fibronectin and/or, PLO+Laminin and/or, PLO+Fibronectin and/or, PDL+Laminin+Fibronectin, etc. The alginate and or hyaluronic acid beads are resuspended in appropriate concentration of coating agents (between 0.00001 mg to 10 mg/ml) and keep it continuous mixing device for 30 min to 24 hrs as per desire coating thickness. Following the incubation period excess coating reagent removed by decanting and the coated beads are wash/rinse three-four times with PBS/HBSS/DPBS or culture medium. These multilayered beads are then subjected for the streptavidinylation process as defined earlier.

In some embodiments the disclosed methods of preparation of microcapsules/microbeads alginate can be replace with hyaluronic acid, hyaluronic acid, chitosan, agarose, and dextran, and proteins, such as gelatin and collagen, alone or in combination. Second or multilayer can be of PDL and/or, PLL and/or, PLO and/or, Collagen and/or, Gelatin and/or, Laminin and/or, Fibronectin and/or, PDL+Laminin and/or, PDL+Fibronectin and/or, PLO+Laminin and/or, PLO+Fibronectin and/or, PDL+Laminin+Fibronectin, etc.

### Example 3: Example Protocol for Streptavidinylation of Alginate—PLO Microbeads

### Preparation of Alginate—PLO Microbeads

To prepare PLO-coated microcapsules first the alginate microbead were fabricated by extruding a solution of 1.5% (w/v) sodium alginate through a 400 μm diameter nozzle of the Buchi B-395 Pro Encapsulator. The droplets were expelled into a 100 mMCaCl2 crosslinking solution and allowed to incubate for fifteen minutes. The microbeads were washed thrice with a solution of 22 mM CaCl2 and 0.9% NaCl for two minutes each. The PLO layer was generated using 0.1% (w/v) solution of PLO in normal saline and rocked for thirty minutes, resulting in the formation of a PLO coating on the microbeads. Three washes were performed with 22 mM CaCl2 and 0.9% NaCl solution to remove residual PLO.

### Streptavidinylation of Alginate-PLO (AL-PLO) Microbeads

Coupling of streptavidin to AL-PLO microbead is composed of three steps.

Step-1, preparation of maleimide-streptavidine complexes (SA-SMCC); Step-2, AL-PLO microbeads reduction by Traut's reagent; and Step-3, Loading of maleimide SA onto Reduced-AL-PLO microbeads (SA-AL-PLO microbeads). Steps 1 and 2 are processed simultaneously.

Step 1: Preparation of Maleimide-SA Complexes (SA-SMCC): Streptavidin is dissolved at 1-10 mg/mL in Hank's Balance Salt Solution (HBSS) and Sulfo-SMCC (S-SMCC) is dissolved separately at 1-10 mg/ml in pre-warmed DPBS (37° C.). Sulfo-SMCC [100 UL at 1-10 mg/mL] is added to each 1-10 mg/ml of streptavidin solution and mixed for 2 hours at room temperature. Un-reacted S-SMCC and free sulfo-N-hydroxsuccinamide is removed by filtration under sterile conditions using YM-10 Micron centrifugal filter devices as per manufacturers guidelines. Following the washing steps the concentration of the SA-SMCC was adjusted to 2-20 mg/ml using sterile HBSS.

Step 2: AL-PLO microbead reduction by Traut's reagnet: AL-PLO microbeads are washed twice with cold HBSS. Discard as much supernatant as possible and disturb the pellet thoroughly. Add 2 ml 1-10 mM sterile filtered Traut's reagent solution per 1-10×10³ AL-PLO microbead, incubate it at RT for 1-10 hrs in dark with continuous mixing. At the end of incubation, wash the AL-PLO microbeads twice with HBSS to remove excess Traut's reagent. Discard the supernatant and re-suspend the AL-PLO microbeads in HBSS at a density of 1-5×10²/ml.

Step 3: Loading of maleimide SA onto reduced-AL-PLO microbeads (SA-AL-PLO microbeads): In a sterile polypropylene tube, mix 1-10 mg of SA-SMCC complex (prepared in step-1) per 1-10×10² reduced AL-PLO microbeads (prepared in step-2), mix well, protect from excessive sunlight with (aluminum foil or paper) and place on an end-to-end rotator at room temperature for 1 hour. At the end of 1 hour, wash the streptavidin coupled AL-PLO microbeads thrice with HBSS to remove excess SA-SMCC. Discard the supernatant and re-suspend the AL-PLO microbeads in HBSS at a density of 1-5×10²/ml.

### Confirm RBC Streptavidinylation by Immuno-Fluorescence Staining

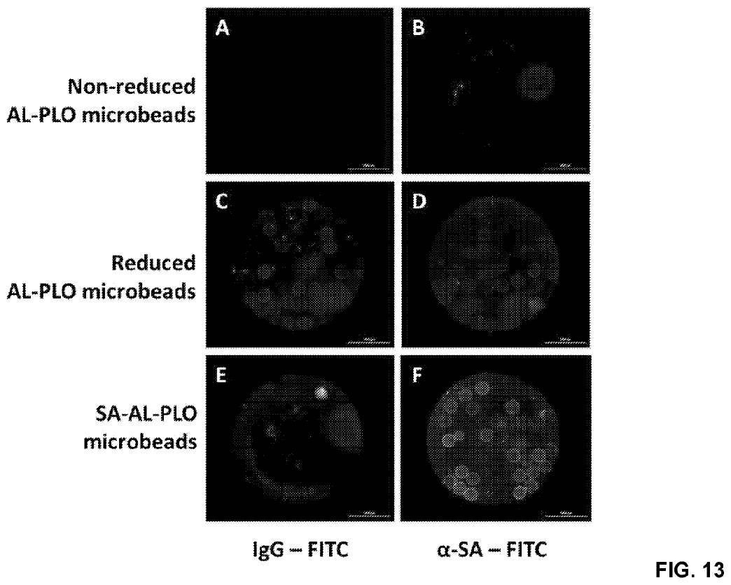

Transfer 1-5×10² AL-PLO microbeads streptavidin conjugates (SA-AL-PLO) and reduced AL-PLO microbeadsin to 24-well tissue culture plates containing 1 mL of HBSS buffer. Add 10-20 μL of FITC anti SA antibody into appropriate wells and mix well. Incubate at 4° C. for 20 minutes. Wash the beads and discard the supernatant and resuspend in 1 mL of HBSS buffer. Immunofluorescent images were captured at 4× magnification on the Lionheart™ FX Automated Microscope (BioTek Instruments Inc., Winooski, VT) using the Gen5 Image+ software, which is a part of the Augmented Microscopy™ suite from BioTek. FIG. 13 shows the immunofluorescence (IF) staining of alginate-PLO (AL-PLO) microbeads. Panel A, C and E are stained with isotype control antibody conjugated with FITC; Panel B, D and F are stained with anti-streptavidin (α-SA) antibody conjugated with FITC. Panel A & B are non-reduced AL-PLO microbeads; Panel C & D are AL-PLO microbeads reduced by Traut's reagent; Panel E & F are streptavidinylated-AL-PLO microbeads.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A cell-targeting complex, comprising a non-nucleated or nucleated cell comprising cell surface proteins that have been thiolated and coated with a streptavidin, avidin, or a derivative or analogue thereof with high affinity for biotin, using a sulfo-SMCC linker, and then coated with one or more biotinylated antibodies.

2. The cell-targeting complex of claim 1, wherein the cell is a red blood cell (RBC) or platelet.

3. A cell-targeting complex, comprising a multilayer alginate hydrogel beads comprising a cell surface that has been coated with a polyanionic protein using a polycation selected from the group consisting of as poly-D-lysine (PDL), poly-L-lysine (PLL), poly-L-ornithine (PLO), and any combination thereof, wherein the coated polyanionic protein is thiolated and streptavidinylated with a sulfo-SMCC linker, and then coated with one or more biotinylated antibodies.

4. The cell-targeting complex of claim 3, wherein the polyanionic protein is selected from the group consisting of collagen, gelatin, laminin, fibronectin, or any combination thereof.

5. The cell-targeting complex of claim 1, wherein the cell surface proteins or coated polyanionic proteins have been thiolated with Traut's reagent (2-Iminothiolane).

6. The cell-targeting complex of claim 1, wherein the one or more biotinylated antibodies comprise anti-CD3 and anti-CD28 antibodies.

7. The cell-targeting complex of claim 6, wherein the cell-targeting complex is loaded with cytokines and/or growth factors configured to enhance activation and proliferation of T cells.

8. The cell-targeting complex of claim 1, wherein the one or more biotinylated antibodies bind tumor antigens.

9. The cell-targeting complex of claim 8, wherein the cell-targeting complex is loaded with costimulatory molecules, death receptors, chemokines, and/or cytokines configured to kill cancer cells.

10. A method for activating an expanding T cells in vitro, comprising contacting the T cells with the cell-targeting complex of claim 6.

11. A method for treating cancer in a subject, comprising administering to the subject an effective amount of the cell-targeting complex of claim 8.

12. A method for transporting an agent in a system to a cell, the method comprising loading the cell-targeting complex of claim 1 with the agent, and contacting the system with the loaded cell-targeting complex.

13. The method of claim 12, wherein the agent is oxygen, a free radical enzyme, a nutrient, or any combination thereof.

14. The method of claim 13, wherein the cell-targeting complex is pre-loaded with an artificial oxygen carrier and then exposed to oxygen prior to or after contact with the system.

15. The method of claim 14, wherein the artificial oxygen carrier comprises perfluorocarbon, HEMOGLOBIN-BASED OXYGEN CARRIERS, X80, FC75, FC43, FC17 (perfluorotetrahydrofuran, perfluorotributylamine), perfluorodecaline, or perfluoromethyl decaline.

16. The method of claim 12, wherein the agent is a viral vector.

17. The method of claim 12, wherein the system is a human or animal subject.

18. A method for expanding, differentiating, or killing a cell in a culture system, comprising culturing the cell in the presence of the cell-targeting complex of claim 1.

19. The method of claim 18, wherein the cell-targeting complex is configured as a feeder layer for the cell.
